(19) United States
(12) Patent Application Publication
(10) Pub. No.: US 2022/0406450 A1
(43) Pub. Date: Dec. 22, 2022

(54) METHODS OF SLEEP STAGE SCORING WITH UNSUPERVISED LEARNING AND APPLICATIONS OF SAME

(71) Applicants: NORTHWESTERN UNIVERSITY, Evanston, IL (US); ANN AND ROBERT H. LURIE CHILDREN'S HOSPITAL OF CHICAGO, Chicago, IL (US)

(72) Inventors: Claus-Peter Richter, Skokie, IL (US); Minh Ha Tran, Laramie, WY (US); Bharat Bhushan, Chicago, IL (US)

(21) Appl. No.: 17/774,535

(22) PCT Filed: Nov. 23, 2020
(86) PCT No.: PCT/US2020/061739

§ 371 Date: May 5, 2022

Related U.S. Application Data
(60) Provisional application No. 62/938,999, filed on Nov. 22, 2019.

(51) Int. Cl. G16H 40/63

(52) U.S. Cl. CPC G16H 40/63

(57) ABSTRACT

The invention relates to a novel method for scoring sleep stages of a mammal subject, comprising extracting features from polysomnography data using the continuous wavelet transform; grouping the extracted features into clusters that are assigned to different sleep stages; and scoring the sleep stages based on the clusters. The method does not require prior visual knowledge of sleep stages nor supervised training.

### CROSS-REFERENCE TO RELATED PATENT APPLICATION

This application claims priority to and the benefit of U.S. provisional patent application Ser. No. 62/938,999, filed Nov. 22, 2019, which is incorporated herein in its entirety by reference.

### STATEMENT AS TO RIGHTS UNDER FEDERALLY-SPONSORED RESEARCH

This invention was made with government support under HL119810 awarded by the National Institutes of Health. The government has certain rights in the invention.

### FIELD OF THE INVENTION

The invention relates generally to data processing, and more particularly to methods of artificial intelligence and automatic computer scoring of sleep stages from data acquired during PSG and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Sleep plays a vital role in good health and well-being throughout our life. It is not surprising that sleep is often sacrificed when people are pressed for time. However, the lack of sleep has been discussed as the underlying mechanism for many diseases, including obesity, diabetes, cardiovascular diseases and hypertension, and immune dysfunction. Since medical conditions resulting from lack of sleep are not immediate, it is difficult to prove the causality of sleep deprivation and the disease.

Normal sleep is characterized by sleep stages, which are typically defined by the neural activity of the brain and by the activity of peripheral muscles. The stages are called awake, non-rapid eye movement sleep divided into stage N1, stage N2, and stage N3, and the rapid eye movement (REM) sleep. In 1937, Loomis discovered distinct visual features such as sleep spindles, K-complex, and delta waves in the electroencephalogram (EEG) of individuals during night sleep (Loomis, Harvey, and Hobart, 1937). He also discovered that those visual features exhibit cyclic phases throughout the entire sleep study. In 1968, Rechtschaffen and Kales classified sleep into five different stages (plus a wake stage) that can be identified using visual markers (Silber et al., 2007). Their manual, often called the R & K sleep manual, which remained the standard for identifying sleep stages until it was replaced in 2007 by a new standard set up by the American Academy of Sleep Medicine (AASM) (Berry et al., 2013). The AASM standard classified sleep into four stages plus a wake stage, the assessment of which is decided visually using the electroencephalogram (EEG), electrooculogram (EOG), and electromyogram (EMG) of individuals. Using the traces of the neurological recording the wake stage or wakefulness is characterized by the presence of alpha (8-13 Hz) and beta (18-25 Hz) waves dominating the occipital EEG. Other features of the wake stage include rapid eye movements and/or blinking. Stage N1 is often described as the transitional stage between the wake stage and stage N2 and is characterized by a transition from a typical alpha rhythm in the EEG recordings into low-amplitude mixed frequency (LAMF, 4-7 Hz) signals. Other markers include slow eye movements and a decrease in the EMG tone. Sleep stage N2 is characterized by the existence of K-complexes and sleep spindles. K-complex is defined as a sharp vertex wave with highest amplitude in the frontal region. Sleep spindle is defined a train of 11-16 Hz sinusoidal wave most distinct in the central region. Sleep stage N3 is characterized by slow delta (0-2 Hz) waves dominating the frontal region. The delta waves are often large in amplitude (>75 mV). Sleep stage R is characterized by LAMF and/or rapid eye movements. Often, there exist sawtooth-like waves in the 2-8 Hz range.

In the clinic, highly trained professionals (usually MDs) identify and visually score sleep stages. This process, also known as “sleep scoring” is done according to standard guidelines released by the AASM. The sleep medicine professional scores each 30 seconds epoch of a full sleep cycle. Since the scoring is mostly visual and relies on intuitions and personal training, the inter-scorer and intra-scorer accuracy is not very high. For example, scoring of 72 subjects by two experienced professionals using the AASM standard only yield an 82% agreement between the two scorers (Danker-Hopfe t al., 2009). Consequently, every 5 epochs or every 2.5 minutes, the scorers disagree. If the average subject sleeps for 7.5 hours, two professionals would disagree, on average, 90 minutes of sleep time about which sleep stages should the subject be in. To fasten the scoring process, methods have been developed to automatically score sleep stages. A listing of some automatic scoring algorithm is shown in Table 1.

TABLE 1
This table summarizes approaches to support the scoring of sleep studies by the use
of computers. Hereby simple techniques to extract frequency features have been used.

| | | Channel | Number of Sleep Stages | | | | |
|---|---|---|---|---|---|---|---|
| Author | Method | Number | 6-class | 5-class | 4-class | 3-class | 2-class |
| Kang et al. 2017 | hidden Markov model, | Fpz-Cz, | | 65.03 | | | |
| | spectral features | F3-A2 | | | | | |
| Supratac et al. 2017 | deep learning | Pz-Oz | | 79.8 | | | |
| | | Fpz-Cz | | 82 | | | |
| Zhu et al. 2014 | difference visibilty | Pz-Oz | 87.5 | 88.9 | 89.3 | 92.6 | 97.9 |
| | graph, SVM | | | | | | |
| Hassan et al. 2016 | statistical features | Pz-Oz | 88.6 | 90.1 | 91.2 | 93.5 | 97.7 |
| | and AdaBoost | | | | | | |
| Hassan et al. 2016 | statistical features | Pz-Oz | 86.8 | 90.6 | 92.1 | 94.1 | 99.4 |
| | and Bagging | | | | | | |
| Hassan et al. 2017 | (EEMD and RUSBoost | Pz-Oz | 88.07 | 83.49 | 92.66 | 94.23 | 98.15 |
| Liang et al. 2012 | entropy feature and | Pz-Oz | | 83.6 | | | |
| | LDA | | | | | | |
| Hsu et al. 2013 | energy feature and | Fpz-Cz | | 87.2 | | | |
| | neural network | | | | | | |
| Doroshenkov et al. | Hidden Markov Model | Pz-Oz, | 61.08 | | | | |
| 2007 | | Fpz-Cz | | | | | |
| Sharma et al. 2017 | TBTFL, wavelet, SVM | Pz-Oz | 91.5 | 91.7 | 92.1 | 93.9 | 98.3 |
| Hassan et al. 2018 | Wavelet transform, | Pz-Oz | 89.3 | 90.8 | 91.55 | 93.9 | 97.18 |
| | Bagging | | | | | | |
| Hassan et al. 2017 | AdaBoost, NIG | Pz-Oz | 90.01 | 91.36 | 92.46 | 94.83 | 98.01 |
| | parameters | | | | | | |
| Hassan et al. 2016 | Wavelet transform, | Pz-Oz | 90.3 | 91.5 | 92.1 | 94.8 | 97.5 |
| | random forest | Fpz-Cz | 87.1 | 88.5 | 90.6 | 92.3 | 96.5 |
| Ghasemzadeh et al. | D3TDWT transform, | Pz-Oz | 92.6 | 93.71 | 94.53 | 96.55 | 99.03 |
| 2019 | LSTAR model, SVM | Fpz-Cz | 92.98 | 93.89 | 94.72 | 96.5 | 98.75 |
| | | both | 93.92 | 94.83 | 95.75 | 97.38 | 99.2 |

The majority of algorithms require extracting multiple features (up to 30 or more) and supervised learning method with the training and testing set often from the same dataset of patients. More recently, more sophisticated and complex models were applied to score sleep studies. The later require extensive training to score stages.

Therefore, a heretofore unaddressed need exists in the art to address the aforementioned deficiencies and inadequacies.

### SUMMARY OF THE INVENTION

One of the objectives of this invention is to provide a method/algorithm for scoring sleep stages that extract only 12 features total from the EEG leads and requires no prior information nor training dataset from other patients. Instead, the method/algorithm relies on clustering methods to cluster accurately sleep stages in their multi-directional space. The method/algorithm is a robust approach of pattern recognition, which is competitive with professional scorers using as little as 2 leads to record brain activity instead of using several leads in the later method mentioned above. The sleep stages includes awake, non-rapid eye movement sleep divided into N1, N2, and N3 stages, and rapid eye movement (REM) sleep.

In one aspect of the invention, the method for scoring sleep stages of a mammal subject, comprising extracting features from polysomnography (PSG) data; grouping the extracted features into clusters that are assigned to different sleep stages; and scoring the sleep stages based on the clusters. The mammal subject is a human subject or a non-human subject.

In one embodiment, the method further comprises, prior to said extracting step, acquiring the PSG data by electroencephalogram (EEG) leads placed on predetermined positions on the mammal subject.

In one embodiment, the PSG data is pre-acquired by EEG leads placed on predetermined positions on the mammal subject.

In one embodiment, said extracting step is performed with a continuous wavelet transform (CWT).

In one embodiment, said extracting step comprises separating each signal recorded with an EEG lead into epochs of a period of time without overlaps; applying the CWT onto each epoch using a mother wavelet to obtain a CWT coefficient matrix; dividing the obtained CWT coefficient matrix into six frequency bands; and summing up the CWT coefficient matrix of each frequency band over the entire sleep epoch to obtain a feature corresponding to said frequency band for said EEG lead, wherein in the end, each epoch taken from said EEG lead has six features extracted.

In one embodiment, said six frequency bands are 0-1 Hz, 1-2 Hz, 2-4 Hz, 4-8 Hz, 8-16 Hz, and 16-32 Hz.

In one embodiment, the mother wavelet is a Morlet wavelet.

In one embodiment, the period of time is in a range of about 20-40 seconds.

In one embodiment, said grouping step is performed with unsupervised learning.

In one embodiment, said grouping step comprises identifying a wake stage; identifying an N1 stage; identifying REM and N2 stages; and identifying an N3 stage.

In one embodiment, said detecting the arousal or sudden movements comprises eliminating outliers using a density-based spatial clustering of applications with a noise (db-scan) algorithm.

In one embodiment, said eliminating the outliers comprises applying the db-scan) algorithm on the frequency features extracted from the PSG data acquired from a occipital (O1-A2) lead, with five frequency features at frequencies 2-4 Hz, 4-8 Hz, 8-16 Hz, and 16-32 Hz, respectively.

In one embodiment, a epsilon distance and nminpoints are adapted in the db-scan algorithm, such that when a group of minimum points are at most epsilon distance apart from each other's, the db-scan algorithm groups them into one cluster, and when there exist any points that are beyond the epsilon distance apart from any available clusters, the db-scan algorithm classifies them as the outliers, where the epsilon distance is a distance used to locate the points in the neighborhood of any point, and the nminpoints are the minimum number of points required to form a dense region.

In one embodiment, said five frequency features are z-score normalized using the epsilon distance of 0.7 and the nminpoints of 15, wherein any outliers registered from the step are scored as a wake stage and discarded before moving on to the next step.

In one embodiment, said identifying the wake stage is performed with hierarchical clustering using a Ward's method, by starting with each point as a cluster of its own; merging two clusters that are the most similar into a new cluster; and repeating the staring and merging steps until there is only one cluster, where the Ward's method groups the clusters that minimized the variance.

In one embodiment, said identifying the wake-sleep stage is performed with three frequency features at frequencies 4-8 Hz, 8-16 Hz, and 16-32 Hz, respectively, extracted from the PSG data acquired from the O1-A2 lead, so as to differentiate awake from sleep.

In one embodiment, said identifying the N1 stage is performed by examining visually cluster groups of the N1 and N2 stages to determine if there are enough points in the N1 stage to classify it as a separated stage; and with insufficient number of points in the N1 stage, marking any transitional points between the wake stage to the N2 stage as the N1 stage.

In one embodiment, said identifying the REM and N2 stages is performed with hierarchical clustering using Ward's criteria with three frequency features at frequencies 2-4 Hz, 4-8 Hz, and 8-16 Hz, respectively, extracted from the PSG data acquired from a frontal (F3-A2) lead, so as to differentiate the REM and N2 stages.

In one embodiment, said identifying the N3 stage is performed with hierarchical clustering using Ward's criteria four frequency features at frequencies 0-1 Hz, 1-2 Hz, 2-4 Hz, and 4-8 Hz, respectively, extracted from the PSG data acquired from the F3-A2 lead, so as to differentiate stage N3 from the other sleep stages.

In one embodiment, said grouping step further comprises smoothing the sleep stages to increase reproducibility of the scoring, which eliminates any outliers that are 2 or less epochs outside of their surrounding neighbors.

In one embodiment, the method further comprises comparing scores of the sleep stages to that given by a professional; and calculating accuracy that is the number of true positive and true negative over the total number of observations, precision that is a ratio of correctly predicted positive observations to the total predicted positive observations, and sensitivity that is a ratio of correctly predicted positive observations to the all observations in actual class.

In another aspect, the invention relates to a non-transitory tangible computer-readable medium storing instructions which, when executed by one or more processors, cause a system to perform a method for scoring sleep stages of a mammal subject, comprising extracting features from the PSG data; grouping the extracted features into clusters that are assigned to different sleep stages; and scoring the sleep stages based on the clusters.

In one embodiment, said extracting step comprises separating each signal recorded with an EEG lead into epochs of a period of time without overlaps; applying the CWT onto each epoch using a mother wavelet to obtain a CWT coefficient matrix; dividing the obtained CWT coefficient matrix into six frequency bands; and summing up the CWT coefficient matrix of each frequency band over the entire sleep epoch to obtain a feature corresponding to said frequency band for said EEG lead, wherein in the end, each epoch taken from said EEG lead has six features extracted.

In one embodiment, said six frequency bands are 0-1 Hz, 1-2 Hz, 2-4 Hz, 4-8 Hz, 8-16 Hz, and 16-32 Hz.

In one embodiment, the mother wavelet is a Morlet wavelet.

In one embodiment, the period of time is in a range of about 20-40 seconds.

In one embodiment, said grouping step is performed with unsupervised learning.

In one embodiment, said grouping step comprises identifying a wake stage; identifying an N1 stage; identifying REM and N2 stages; and identifying an N3 stage.

In one embodiment, said detecting the arousal or sudden movements comprises eliminating outliers using a db-scan algorithm.

In one embodiment, said eliminating the outliers comprises applying the db-scan) algorithm on the frequency features extracted from the PSG data acquired from the O1-A2 lead, with five frequency features at frequencies 2-4 Hz, 4-8 Hz, 8-16 Hz, and 16-32 Hz, respectively.

In one embodiment, a epsilon distance and nminpoints are adapted in the db-scan algorithm, such that when a group of minimum points are at most epsilon distance apart from each other's, the db-scan algorithm groups them into one cluster, and when there exist any points that are beyond the epsilon distance apart from any available clusters, the db-scan algorithm classifies them as the outliers, where the epsilon distance is a distance used to locate the points in the neighborhood of any point, and the nminpoints are the minimum number of points required to form a dense region.

In one embodiment, said five frequency features are z-score normalized using the epsilon distance of 0.7 and the nminpoints of 15, wherein any outliers registered from the step are scored as a wake stage and discarded before moving on to the next step.

In one embodiment, said identifying the wake stage is performed with hierarchical clustering using a Ward's method, by starting with each point as a cluster of its own; merging two clusters that are the most similar into a new cluster; and repeating the staring and merging steps until there is only one cluster, where the Ward's method groups the clusters that minimized the variance.

In one embodiment, said identifying the wake-sleep stage is performed with three frequency features at frequencies 4-8 Hz, 8-16 Hz, and 16-32 Hz, respectively, extracted from the PSG data acquired from the O1-A2 lead, so as to differentiate awake from sleep.

In one embodiment, said identifying the N1 stage is performed by examining visually cluster groups of the N1 and N2 stages to determine if there are enough points in the N1 stage to classify it as a separated stage; and with insufficient number of points in the N1 stage, marking any transitional points between the wake stage to the N2 stage as the N1 stage.

In one embodiment, said identifying the REM and N2 stages is performed with hierarchical clustering using Ward's criteria with three frequency features at frequencies 2-4 Hz, 4-8 Hz, and 8-16 Hz, respectively, extracted from the PSG data acquired from the F3-A2 lead, so as to differentiate the REM and N2 stages.

In one embodiment, said identifying the N3 stage is performed with hierarchical clustering using Ward's criteria four frequency features at frequencies 0-1 Hz, 1-2 Hz, 2-4 Hz, and 4-8 Hz, respectively, extracted from the PSG data acquired from the F3-A2 lead, so as to differentiate stage N3 from the other sleep stages.

In one embodiment, said grouping step further comprises smoothing the sleep stages to increase reproducibility of the scoring, which eliminates any outliers that are 2 or less epochs outside of their surrounding neighbors.

In one embodiment, the method further comprises comparing scores of the sleep stages to that given by a professional; and calculating accuracy that is the number of true positive and true negative over the total number of observations, precision that is a ratio of correctly predicted positive observations to the total predicted positive observations, and sensitivity that is a ratio of correctly predicted positive observations to the all observations in actual class.

In yet another aspect, the invention relates to a system for scoring sleep stages of a mammal subject, comprising one or more computing devices comprising one or more processors; and a non-transitory tangible computer-readable medium storing instructions which, when executed by the one or more processors, cause the one or more computing devices to perform a method for scoring sleep stages of a mammal subject, comprising extracting features from the PSG data; grouping the extracted features into clusters that are assigned to different sleep stages; and scoring the sleep stages based on the clusters.

In one embodiment, said extracting step comprises separating each signal recorded with an EEG lead into epochs of a period of time without overlaps; applying the CWT onto each epoch using a mother wavelet to obtain a CWT coefficient matrix; dividing the obtained CWT coefficient matrix into six frequency bands; and summing up the CWT coefficient matrix of each frequency band over the entire sleep epoch to obtain a feature corresponding to said frequency band for said EEG lead, wherein in the end, each epoch taken from said EEG lead has six features extracted.

In one embodiment, said six frequency bands are 0-1 Hz, 1-2 Hz, 2-4 Hz, 4-8 Hz, 8-16 Hz, and 16-32 Hz.

In one embodiment, the mother wavelet is a Morlet wavelet.

In one embodiment, the period of time is in a range of about 20-40 seconds.

In one embodiment, said grouping step is performed with unsupervised learning.

In one embodiment, said grouping step comprises identifying a wake stage; identifying an N1 stage; identifying REM and N2 stages; and identifying an N3 stage.

In one embodiment, said detecting the arousal or sudden movements comprises eliminating outliers using a db-scan algorithm.

In one embodiment, said eliminating the outliers comprises applying the db-scan) algorithm on the frequency features extracted from the PSG data acquired from the O1-A2 lead, with five frequency features at frequencies 2-4 Hz, 4-8 Hz, 8-16 Hz, and 16-32 Hz, respectively.

In one embodiment, a epsilon distance and nminpoints are adapted in the db-scan algorithm, such that when a group of minimum points are at most epsilon distance apart from each other's, the db-scan algorithm groups them into one cluster, and when there exist any points that are beyond the epsilon distance apart from any available clusters, the db-scan algorithm classifies them as the outliers, where the epsilon distance is a distance used to locate the points in the neighborhood of any point, and the nminpoints are the minimum number of points required to form a dense region.

In one embodiment, said five frequency features are z-score normalized using the epsilon distance of 0.7 and the nminpoints of 15, wherein any outliers registered from the step are scored as a wake stage and discarded before moving on to the next step.

In one embodiment, said identifying the wake stage is performed with hierarchical clustering using a Ward's method, by starting with each point as a cluster of its own; merging two clusters that are the most similar into a new cluster; and repeating the staring and merging steps until there is only one cluster, where the Ward's method groups the clusters that minimized the variance.

In one embodiment, said identifying the wake-sleep stage is performed with three frequency features at frequencies 4-8 Hz, 8-16 Hz, and 16-32 Hz, respectively, extracted from the PSG data acquired from the O1-A2 lead, so as to differentiate awake from sleep.

In one embodiment, said identifying the N1 stage is performed by examining visually cluster groups of the N1 and N2 stages to determine if there are enough points in the N1 stage to classify it as a separated stage; and with insufficient number of points in the N1 stage, marking any transitional points between the wake stage to the N2 stage as the N1 stage.

In one embodiment, said identifying the REM and N2 stages is performed with hierarchical clustering using Ward's criteria with three frequency features at frequencies 2-4 Hz, 4-8 Hz, and 8-16 Hz, respectively, extracted from the PSG data acquired from the F3-A2 lead, so as to differentiate the REM and N2 stages.

In one embodiment, said identifying the N3 stage is performed with hierarchical clustering using Ward's criteria four frequency features at frequencies 0-1 Hz, 1-2 Hz, 2-4 Hz, and 4-8 Hz, respectively, extracted from the PSG data acquired from the F3-A2 lead, so as to differentiate stage N3 from the other sleep stages.

In one embodiment, said grouping step further comprises smoothing the sleep stages to increase reproducibility of the scoring, which eliminates any outliers that are 2 or less epochs outside of their surrounding neighbors.

In one embodiment, the method further comprises comparing scores of the sleep stages to that given by a professional; and calculating accuracy that is the number of true positive and true negative over the total number of observations, precision that is a ratio of correctly predicted positive observations to the total predicted positive observations, and sensitivity that is a ratio of correctly predicted positive observations to the all observations in actual class.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows schematically a flowchart of a method for scoring sleep stages of a mammal subject according to embodiments of the invention.

FIG. 2 shows features extraction from the dataset according to embodiments of the invention. The CWT with the Morlet mother wavelet is applied to the recorded trace of the selected lead. The resulting coefficient matrix is divided into six frequency bands. The matrix is summed, which is used as a feature for this lead.

FIG. 3 shows an overview of subjects by number and age according to embodiments of the invention.

FIG. 4 shows scatterplot of points extracted from the F3-A2 lead of one of the patient with OSA according to embodiments of the invention.

FIG. 5 shows overlaying of scorer one's grading on top of FIG. 4, showing a clustering between sleep stages wake (W) and REM according to embodiments of the invention. Notice also the confusion between stage N2 and N3.

FIG. 6 shows simplified model of how the sleep clusters are positioned in relation to the frequency contribution according to embodiments of the invention.

FIG. 7 shows scatter plot of one subject showing very poor clusters of points according to embodiments of the invention. The agreement between scorers for this patient is 62%, the lowest within the dataset of subjects with OSA.

FIG. 8A shows confusion matrix for the patient in FIG. 7 showing the disagreements between both scorers are between stages W, N1, and N2 according to embodiments of the invention.

FIG. 8B shows confusion matrix for the patient in FIG. 5 showing the disagreements between both scorers are between stages N3.

FIG. 9A shows confusion matrix of the computer against scorer 1, stacking all 61 patients with OSA together according to embodiments of the invention.

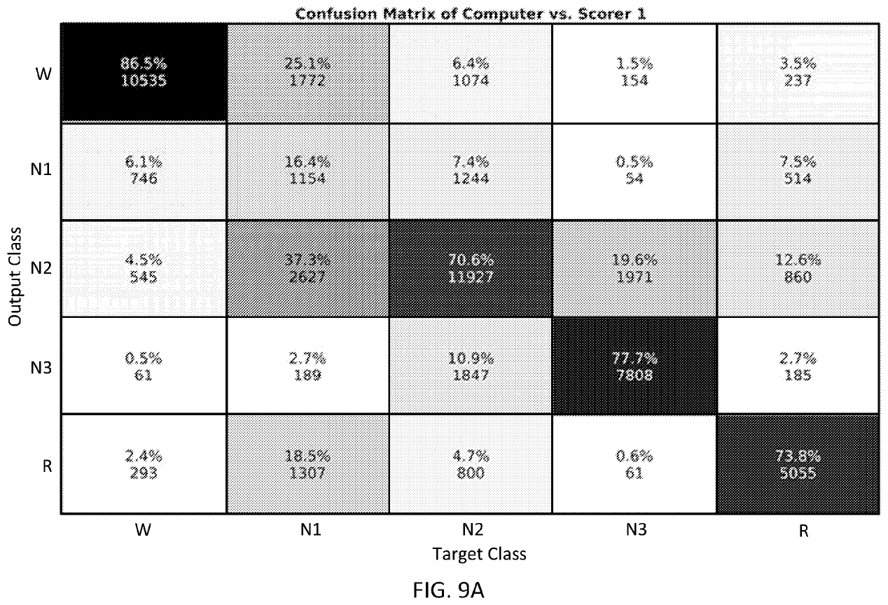

FIG. 9B shows confusion matrix of the computer against scorer 2 stacking all 61 patients with OSA together according to embodiments of the invention.

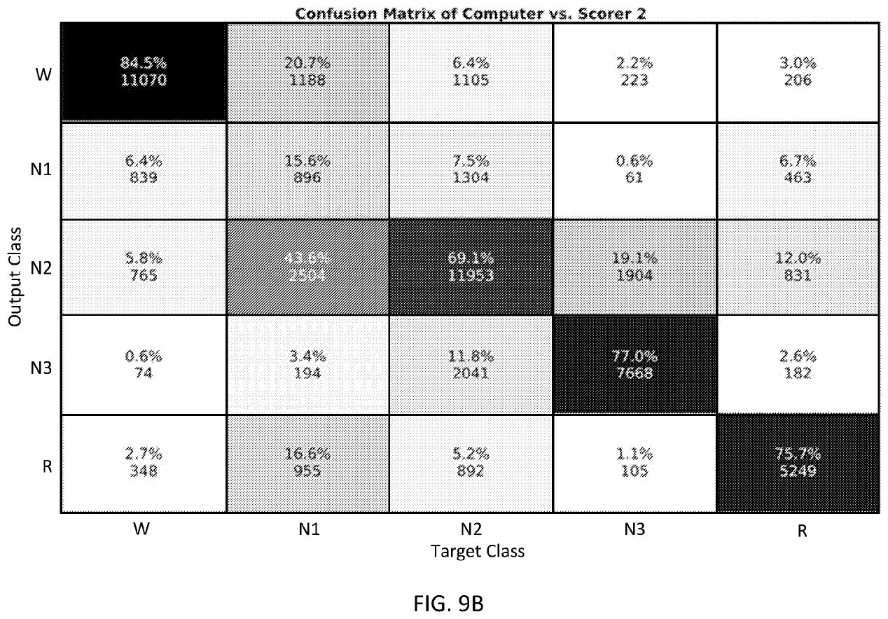

FIG. 10 shows sleep stage percentage contribution, as graded by the algorithm, the first scorer, and the second scorer respectively for all 61 patients with OSA according to embodiments of the invention.

FIG. 11 shows comparison of hypnograms according to embodiments of the invention. Top: Before smoothing stage is applied. Middle: After smoothing stage is applied. Bottom: Professional scorer's data. Accuracy achieved 85% before smoothing and 87% after smoothing. The stages W, N1, N2, N3, and R are respectively numbered 0, 1, 2, 3, and 5 by professionals' standard.

FIG. 12 shows scatterplot of accuracy of scorer 1, scorer 2, and the algorithm according to embodiments of the invention.

### DETAILED DESCRIPTION OF THE INVENTION

The present invention will now be described more fully hereinafter with reference to the accompanying drawings, in which exemplary embodiments of the present invention are shown. The present invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present invention.

It will be understood that, as used in the description herein and throughout the claims that follow, the meaning of “a”, “an”, and “the” includes plural reference unless the context clearly dictates otherwise. Also, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

The methods and systems will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

According to the American Academy of Sleep Medicine (AASM), sleep can be divided into different stages N1, N2, N3, REM and awake. The time spent in each stage as well as the cycling between the stages hold clinical importance. Therefore, it is of utmost importance to score sleep stages correctly. Standard procedure requires sleep professionals to score sleep stages using polysomnography (PSG), also known as sleep study data. These sleep professionals are trained and follow the AASM guidelines to recognize and score visual and temporal features of each sleep stage. However, it has been shown that these sleep professionals' performance has limited inter- and intra-patient reproducibility.

One of the objectives of the invention is to provide a novel computer-based method/algorithm for scoring sleep stages that does not require prior visual knowledge of sleep stages nor supervised training. Instead, the novel algorithm extract features from the PSG data using the continuous wavelet transform, then build clusters of data that are assigned to different sleep stages. When comparing to professional scorers, the algorithm, agrees on average with the professional scorers 80% of the time, which is comparable to how much the scorers agrees with each other, which is 85% of the time on average. Since the outcome of computer scoring is always reproducible independent of how often the sleep study is scored or the location where it is scored, the use of the computer could lead to an increased inter-site and overall rigor and reproducibility of the results among different sleep disorder centers. The inter-scorer variability is also removed.

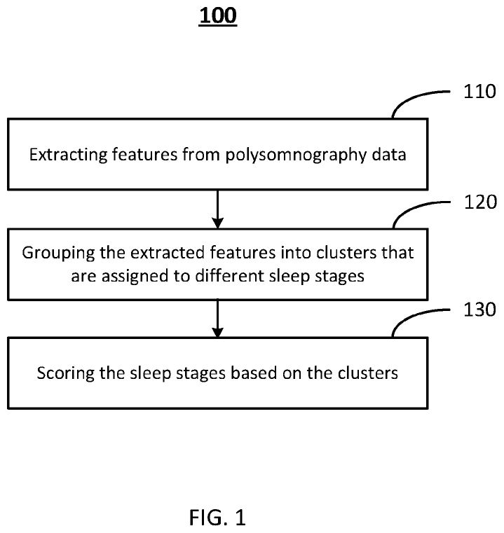

Referring to FIG. 1, the method 100 for scoring sleep stages of a mammal subject, comprises extracting features from the PSG data (at step 110); grouping the extracted features into clusters that are assigned to different sleep stages (at step 120); and scoring the sleep stages based on the clusters (at step 130). The mammal subject is a human subject or a non-human subject.

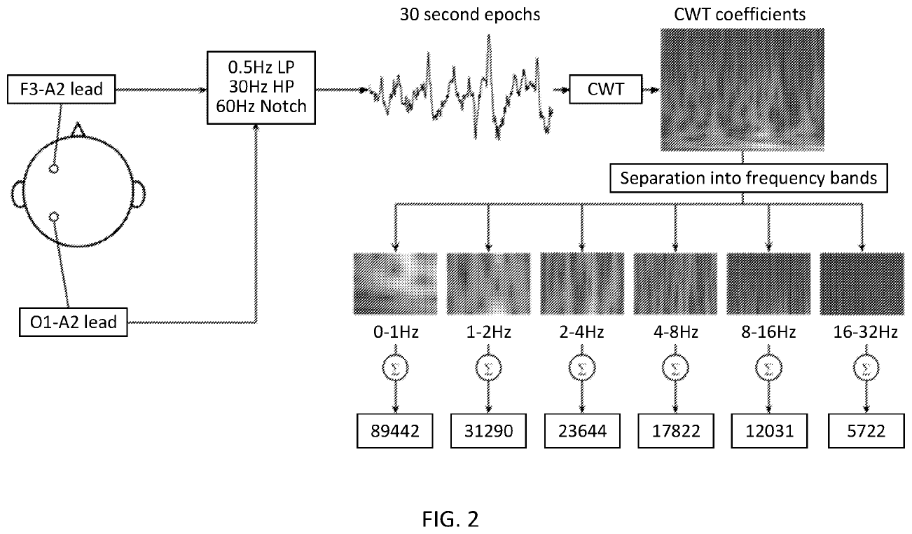

The method may further include, prior to said extracting step, acquiring the PSG data by electroencephalogram (EEG) leads placed on predetermined positions on the mammal subject. According to the invention, the PSG data used for scoring the sleep stages are signals acquired from only two leads: a frontal (F3-A2) lead and an occipital (O1-A2) lead, as shown in FIG. 2. Alternatively, the PSG data is pre-acquired by the F3-A2 lead and the O1-A2 lead.

In one embodiment, said extracting step is performed with a continuous wavelet transform (CWT).

In one embodiment shown in FIG. 2, said extracting step comprises separating each signal recorded with an EEG lead into epochs of a period of time without overlaps; applying the CWT onto each epoch using a mother wavelet to obtain a CWT coefficient matrix; dividing the obtained CWT coefficient matrix into six frequency bands; and summing up the CWT coefficient matrix of each frequency band over the entire sleep epoch to obtain a feature corresponding to said frequency band for said EEG lead, wherein in the end, each epoch taken from said EEG lead has six features extracted. In one embodiment, said six frequency bands are 0-1 Hz, 1-2 Hz, 2-4 Hz, 4-8 Hz, 8-16 Hz, and 16-32 Hz. In one embodiment, the mother wavelet is a Morlet wavelet. In one embodiment, the period of time is in a range of about 20-40 seconds.

In one embodiment, said grouping step is performed with unsupervised learning.

In one embodiment, said grouping step comprises identifying a wake stage; identifying an N1 stage; identifying REM and N2 stages; and identifying an N3 stage.

In one embodiment, said detecting the arousal or sudden movements comprises eliminating outliers using a density-based spatial clustering of applications with a noise (db-scan) algorithm. In the db-scan algorithm, a epsilon distance and nminpoints are adapted such that when a group of minimum points are at most epsilon distance apart from each other's, the db-scan algorithm groups them into one cluster, and when there exist any points that are beyond the epsilon distance apart from any available clusters, the db-scan algorithm classifies them as the outliers, where the epsilon distance is a distance used to locate the points in the neighborhood of any point, and the nminpoints are the minimum number of points required to form a dense region.

In one embodiment, said eliminating the outliers comprises applying the db-scan) algorithm on the frequency features extracted from the PSG data acquired from the O1-A2 lead, with five frequency features at frequencies 2-4 Hz, 4-8 Hz, 8-16 Hz, and 16-32 Hz, respectively.

In one embodiment, said five frequency features are z-score normalized using the epsilon distance of 0.7 and the nminpoints of 15, where any outliers registered from the step are scored as a wake stage and discarded before moving on to the next step. It should be appreciated that other values of the epsilon distance and the nminpoints can also be utilized to practice the invention.

In one embodiment, said identifying the wake stage is performed with hierarchical clustering using a Ward's method, by starting with each point as a cluster of its own; merging two clusters that are the most similar into a new cluster; and repeating the staring and merging steps until there is only one cluster, where the Ward's method groups the clusters that minimized the variance.

In one embodiment, said identifying the wake stage is performed with three frequency features at frequencies 4-8 Hz, 8-16 Hz, and 16-32 Hz, respectively, extracted from the PSG data acquired from the O1-A2 lead, so as to differentiate awake from sleep.

In one embodiment, said identifying the N1 stage is performed by examining visually cluster groups of the N1 and N2 stages to determine if there are enough points in the N1 stage to classify it as a separated stage; and with insufficient number of points in the N1 stage, marking any transitional points between the wake stage to the N2 stage as the N1 stage.

In one embodiment, said identifying the REM and N2 stages is performed with hierarchical clustering using Ward's criteria with three frequency features at frequencies 2-4 Hz, 4-8 Hz, and 8-16 Hz, respectively, extracted from the PSG data acquired from the F3-A2 lead, so as to differentiate the REM and N2 stages.

In one embodiment, said identifying the N3 stage is performed with hierarchical clustering using Ward's criteria four frequency features at frequencies 0-1 Hz, 1-2 Hz, 2-4 Hz, and 4-8 Hz, respectively, extracted from the PSG data acquired from the F3-A2 lead, so as to differentiate stage N3 from the other sleep stages.

In one embodiment, said grouping step further comprises smoothing the sleep stages to increase reproducibility of the scoring, which eliminates any outliers that are 2 or less epochs outside of their surrounding neighbors.

In one embodiment, the method further comprises comparing scores of the sleep stages to that given by a professional; and calculating accuracy that is the number of true positive and true negative over the total number of observations, precision that is a ratio of correctly predicted positive observations to the total predicted positive observations, and sensitivity that is a ratio of correctly predicted positive observations to the all observations in actual class.

In another aspect, the invention relates to a system for scoring sleep stages of a mammal subject. The system includes one or more computing devices comprising one or more processors configured to perform the method for scoring sleep stages as disclosed above. Furthermore, the system also includes a non-transitory tangible computer-readable medium storing instructions which, when executed by the one or more processors, cause the one or more computing devices to perform the method for scoring sleep stages of a mammal subject, as disclosed above.

It should be noted that all or a part of the steps according to the embodiments of the present invention is implemented by hardware or a program instructing relevant hardware. Yet another aspect of the invention provides a non-transitory tangible computer-readable medium storing instructions which, when executed by one or more processors, cause a system to perform the method for scoring sleep stages of a mammal subject, as disclosed above. The computer executable instructions or program codes enable a computer or a similar computing system to complete various operations in the above disclosed method for privilege management. The storage medium/memory may include, but is not limited to, high-speed random access medium/memory such as DRAM, SRAM, DDR RAM or other random access solid state memory devices, and non-volatile memory such as one or more magnetic disk storage devices, optical disk storage devices, flash memory devices, or other non-volatile solid state storage devices.

These and other aspects of the present invention are further described below. Without intent to limit the scope of the invention, examples according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Features Extraction

In certain embodiments, the algorithm for the computer-based scoring of the sleep studies uses the CWT. The wavelet transform is a powerful method to analyze signals due to its ability to be both a time- and frequency-localized transformation. The transform was first conceived as a continuous transform, where a continuous wavelet (called the “mother wavelet”) is scaled and translated over the entire continuous signal, giving a continuous two-dimensional function of inner product values. In certain embodiments, the algorithm uses the “Morlet” mother wavelet. In the past, seizures (Ocak, 2009), sleep onset (Lee, Lee, & Chung, 2014), and sleep spindles (Al-Salman, Li, & Wen, 2019) have been predicted with the CWT.

In certain embodiments, each signal recorded with one lead was separated into epochs of 30 seconds without overlaps. Then, the CWT was applied onto each epoch using the Morlet mother wavelet. The resulting CWT coefficient magnitude was divided into six frequency bands, as shown in FIG. 2, and summed up over the entire sleep epoch. In the end, each epoch taken from each EEG lead has six features total.

### Unsupervised Learning

Arousal Detection: Arousal or sudden movements produce outliers in the datasets, which potentially skew the clustering algorithm. To eliminate outliers, a density-based spatial clustering of applications with noise (db-scan) algorithm is used. The algorithm required two parameters: a neighboring value epsilon and a neighboring number nminpoints. If a group of minimum points are at most epsilon distance apart from each other's, then the algorithm groups them into one cluster. If there exist any points that are beyond epsilon distance apart from any available clusters, then the algorithm classifies them as outliers or noise. In certain embodiments, the db-scan is applied on the frequency features extracted from the O1-A2 lead, because the occipital leads are more susceptible to arousal. Five features 2-4 Hz, 4-8 Hz, 8-16 Hz, and 16-32 Hz are considered. These features were z-score normalized. An epsilon value of 0.7 and a nminpoints value of 15 were used. Any outliers registered from this step were scored as wake stage and discarded before moving on to the next step.

Wake-Sleep Identification: For wake-sleep stage identification, hierarchical clustering using Ward's method was used. The basic of hierarchical clustering was iterative: (1) start with each point as a cluster of its own. (2) Merge two clusters that are the most similar into a new cluster. (3) repeat (1) and (2) until there is only one cluster. The complexity comes from the metrics used to specified distance for merging. Ward's method groups the clusters that minimized the variance. This method worked best on clusters that followed an elliptical distribution when plotted on a multi-dimensional scatter plot. Furthermore, Ward suggested that the algorithm should be used on large (n>100) dataset, which the sleep epochs satisfy (n>700).

Only the O1-A2 lead was used for wake sleep stage identification. Three features 4-8 Hz, 8-16 Hz, and 16-32 Hz were used to differentiate awake from sleep. The data had outliers removed from the previous step in the arousal detection. The data was then log-normalized and then rescaled on a 0-1 scale. Calinski-Harabasz's method of cluster identification was used to provide the optimal amount of clusters present. Hierarchical clustering using Ward's method was used with the optimal clusters number as input. After completing the clustering by the computer, an individual examines the machine clustering visually through a scatterplot and can then decide and assign if the clusters belong to the sleep stages or the wake stage.

N1 Identification: In a healthy individual, stage N1 should take only 5% of the entire sleep stages. This stage has no clear features by which it can be identified and separated from other sleep stages. Therefore, it is hard to identify clusters within the N1 group. As a result, the cluster group N1 and N2 was examined visually to determine if there are enough points in stage N1 to classify it as a separated stage. With insufficient number of points in stage N1, any transitional points between wake stage to stage N2 was marked as stage N1.

REM and N2 Identification: Only the F3-A2 lead was used for REM and N2 identification. Three features 2-4 Hz, 4-8 Hz, and 8-16 Hz were used to differentiate stage REM and N2. The arousal, wake, and N3 stages identified from the previous step were removed. The data was log-normalized, but not rescaled. Hierarchical clustering using Ward's criteria was used for this step. However, the number of clusters was forced to two clusters. An individual who examines the machine clustering visually through a scatterplot can then assign the clusters to the REM or N2 sleep stages.

Stage N3 Identification: Only the F3-A2 lead was used for stage N3 identification. Four features 0-1 Hz, 1-2 Hz, 2-4 Hz, and 4-8 Hz were used to differentiate stage N3 from the other sleep stages. The arousal and wake sleep stages identified from the previous step were removed to improve the quality of the clustering algorithm. The data was log-normalized, but not rescaled. Hierarchical clustering using Ward's criteria was used for this step, similar to how Wake-Sleep was identified in the previous step. Calinski-Harabasz's method was used to provide the optimal amount of clusters present. This optimal number was used as the input to the clustering algorithm. An individual who examines the machine clustering visually through a scatterplot can then assign if the clusters belong to stage N3 or to the other sleep stages.

Smoothing Sleep Stages: Smoothing sleep stages is a method proposed by Liang et al. (Liang et al., 2012) to increase reproducibility of the scoring. Since sleep stages were often unchanged throughout the entire sleep, it appeared better to change “outlier” sleep stages to their proceeding sleep stage. An algorithm to smooth sleep stages was developed. This algorithm eliminates any outliers that are 2 or less epochs outside of their surrounding neighbors.

Comparison Metrics: After clustering, the scoring given by the computer was compared to the scoring given by a professional. Accuracy (the number of true positive and true negative over the total number of observations), precision (ratio of correctly predicted positive observations to the total predicted positive observations), and sensitivity (ratio of correctly predicted positive observations to the all observations in actual class) were calculated.

### Validation

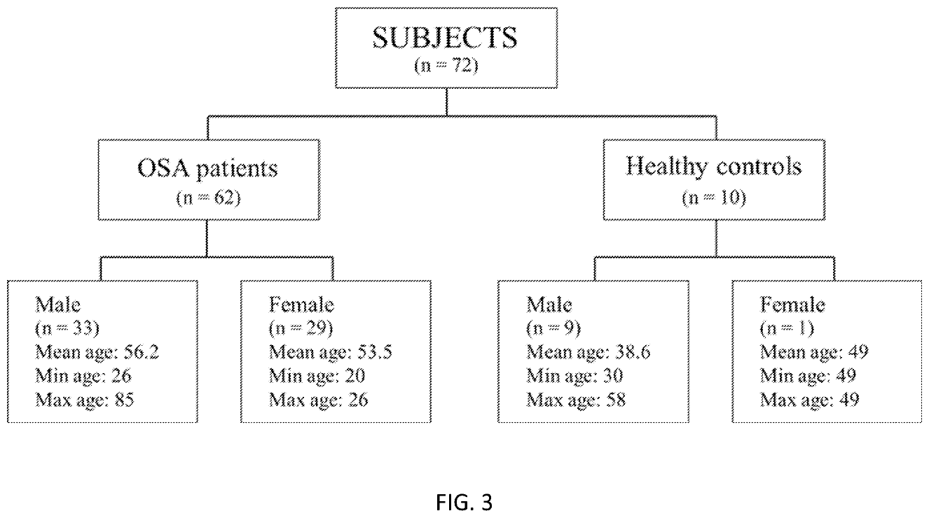

Subjects: The EEG dataset, which is pre-acquired and is publicly available online (https://sleeptight.isr.uc.pt/ISRUC_Sleep/) was used for this study. The recordings originate from 100 adult subjects with evidence for sleep disorders and 10 healthy subjects, which were used as a control group (FIG. 3). Of the 100 adult subjects with evidence for sleep disorders, 62 had obstructive sleep apnea (OSA). All 10 healthy subjects were included in the study. The database contains recordings obtained with 19 channels: two are for the electro-oculograms (EOGs) left and right eye), six are record the electroencephalograms (EEGs: F3-A2, C3-A2, O1-A2, F4-A1, C4-A1, O2-A1), three leads are used for the electromyogram (EMG: chin, left leg, and right leg), one is for the electrocardiogram (ECG), one records the acoustical signal to detect snoring, two leads measure airflow, one registers the abdominal position, one is used for pulse oximetry, and one detects body position (Khalighi, Sousa, Santos, & Nunes, 2016).

Data were acquired using a sampling rate of 200 Hz with 16-bit resolution. The filter settings of the cutoff frequencies were 0.5-30 Hz for EEG/EOG. In the original dataset, the last 25 minutes or 30 epochs of data were not included. Included with the database was the scoring given by two independent professionals for each patient. The professional scores used all the traces acquired during the PSG according to the AASM manual.

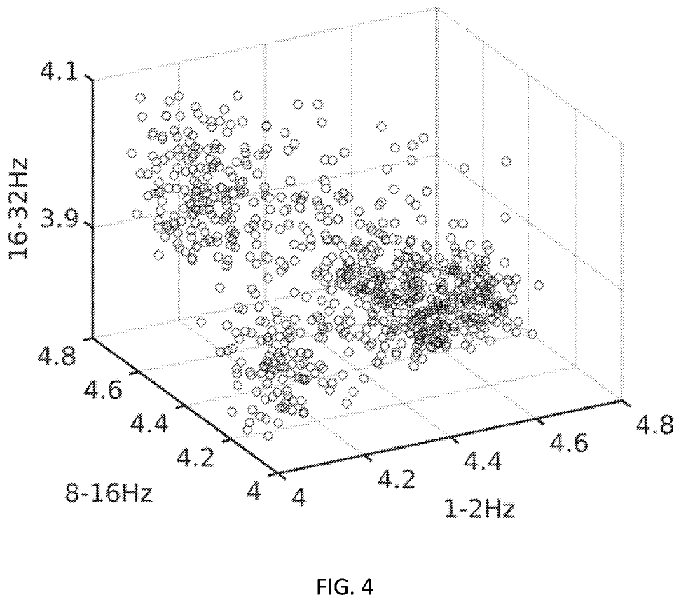

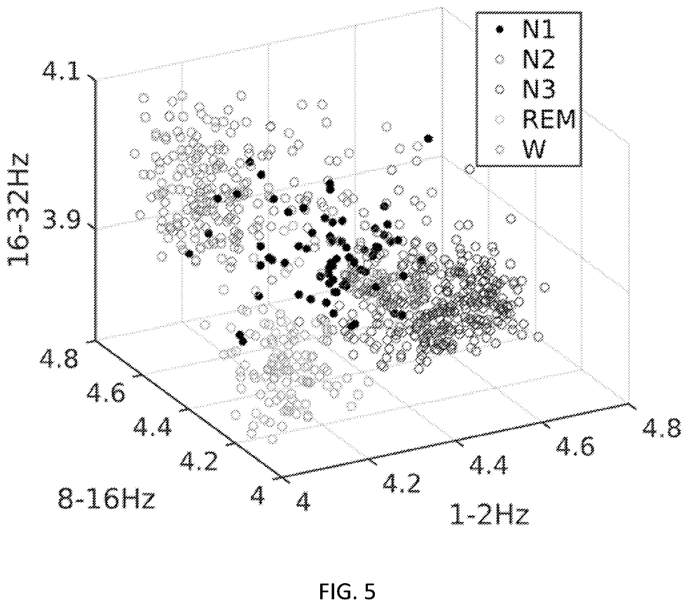

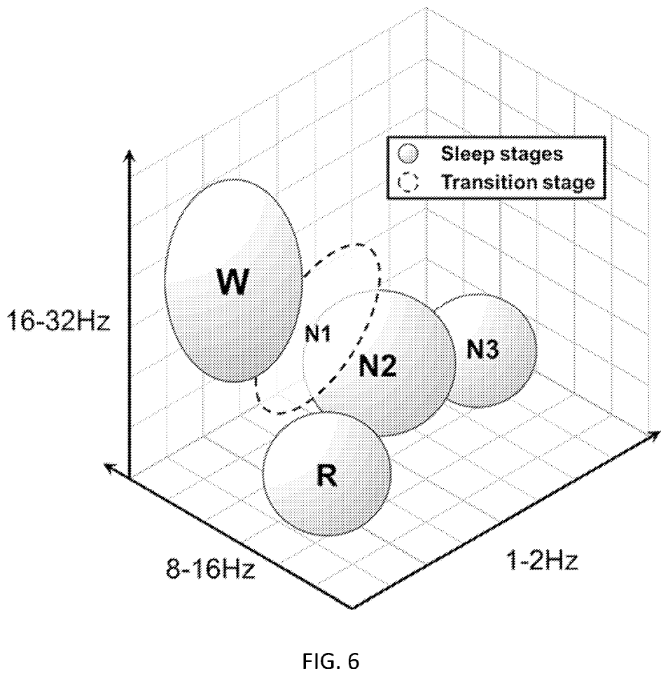

Existence of Clusters: FIG. 4 shows the scatterplot of points using features extracted from the F3-A2 lead on the frequency bands 2-4 Hz, 8-16 Hz, and 16-32 Hz from one of the patient with OSA. FIG. 5 shows the overlay of FIG. 4 with scorer one's opinion on how the scores should be assigned. Both FIGS. 3 and 4 show a specific clustering of points that can be chosen by the algorithm. Notice the position of stage N1 in relation to stage W and stage N2. This agrees with AASM's observation that stage N1 serve as a transition between W and N2 and with the recommendation to score epochs as N1 when attenuation of alpha waves into LAMF wave is recorded. FIG. 6 shows a simplified model of how sleep stages clusters positions in relation to each other's on a scatter plot.

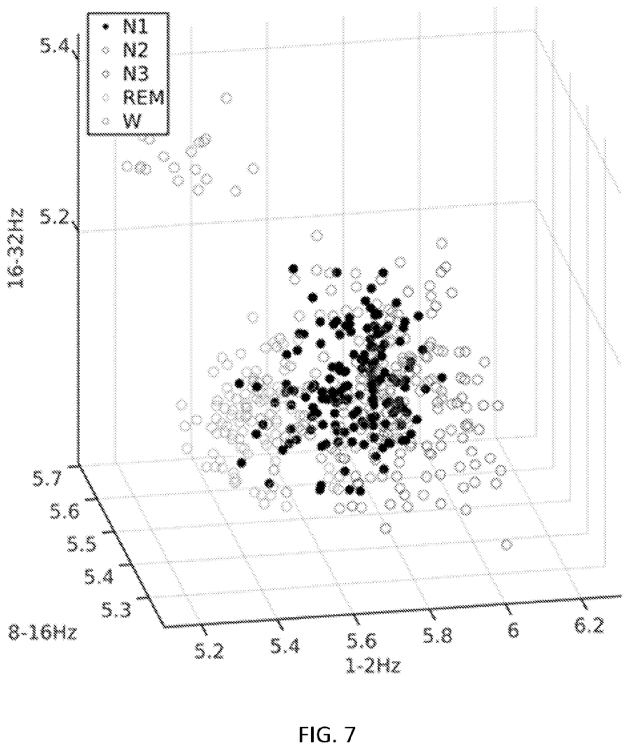

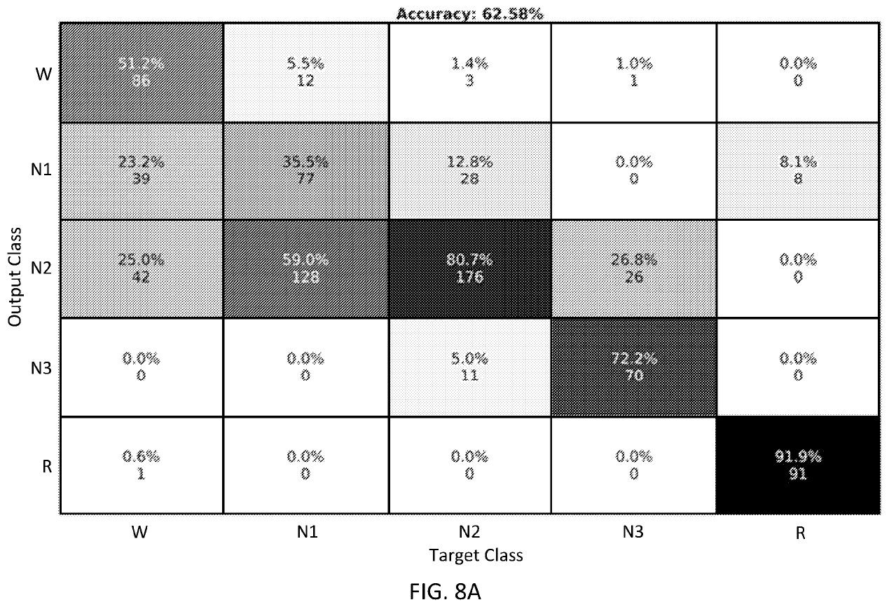

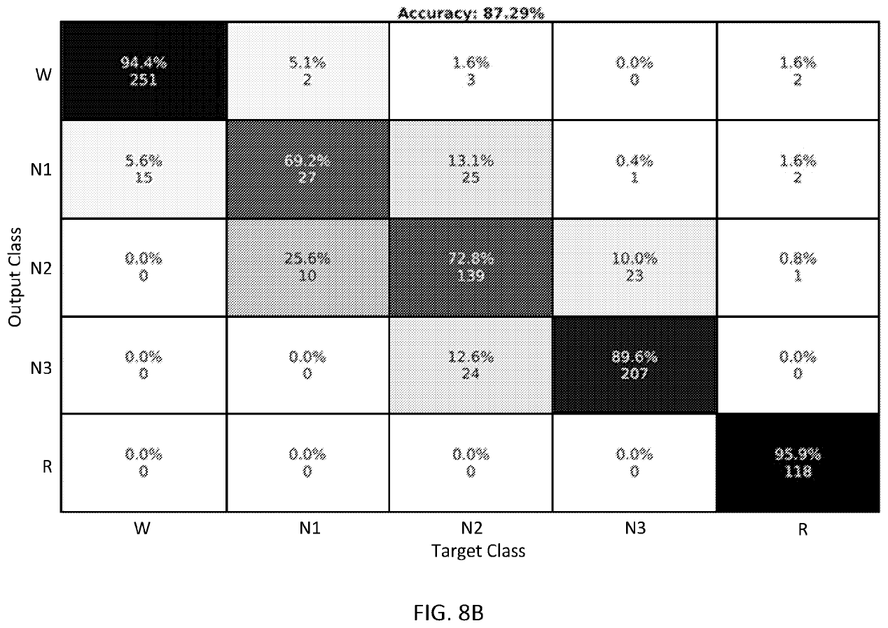

One observation was that more defined clusters of stages correlate well with a higher agreement among the scorers to distinguish between the sleep stages. FIG. 7 shows the scatterplot for one of the patients with only 62% agreements between the scorers. The figure shows poor clustering between stage N1, N2, and N3, and REM stages. As a result, looking at the confusion matrix in FIG. 8A shows strong disagreements between sleep stage N1, N2, and N3.

The scatterplot shown in FIG. 5 for one of the patients with 87% agreements between the scorers was also well clustered. This is the highest agreements between the two scorers within our dataset of adult population. The agreement between scorers can be shown in the confusion matrix shown in FIG. 8B.

Wake-Sleep-N1 Detection: Table 2 shows the accuracy between wake and sleep stages for the 61 patients in the adult population with OSA, along with the scorers' agreements accuracy. Table 3 shows the same values for the 10 healthy individuals. A sleep stage smoothing algorithm was applied. Across all patients with OSA, the algorithm achieved a precision of 73.0±17.7% against scorer 1 and 76.5±17.0% against scorer 2; the algorithm achieved a sensitivity (recall) of 84.0±10.9% against scorer 1 and 82.5±11.5% against scorer 2. Across all healthy individuals, the algorithm achieved a precision of 71.3±16.9% against scorer 1 and 66.9±16.3% against scorer 2; the algorithm achieved a sensitivity (recall) of 86.6±7.1% against scorer 1 and 89.0±7.2% against scorer 2.

TABLE 2
Wake-sleep classification accuracy for the 61 adult patients with OSA.
Wake-Sleep accuracy

| | | Scorer 1 | | Scorer 2 | | Both scorers | |
|---|---|---|---|---|---|---|---|
| Patient | Algorithm | Precision | Sensitivity | Precision | Sensitivity | Precision | Sensitivity |
| 1 | Ward's algorithm | 87.23% | 95.35% | 90.43% | 95.86% | 97.29% | 94.36% |
| 2 | Ward's algorithm | 88.70% | 85.36% | 78.26% | 86.96% | 84.94% | 98.07% |
| 5 | Ward's algorithm | 88.85% | 89.85% | 93.31% | 89.01% | 97.74% | 92.20% |
| 7 | Ward's algorithm | 74.73% | 50.75% | 76.92% | 49.65% | 91.79% | 87.23% |
| 8 | Ward's algorithm | 80.98% | 86.01% | 83.41% | 82.61% | 95.34% | 88.89% |
| 9 | Correlation K-means | 50.53% | 78.51% | 46.81% | 73.33% | 62.81% | 63.33% |
| 10 | Correlation K-means | 91.95% | 94.48% | 89.60% | 96.74% | 93.79% | 98.55% |
| 11 | Correlation K-means | 89.90% | 83.38% | 99.02% | 68.31% | 99.40% | 73.93% |
| 15 | Correlation K-means | 77.63% | 89.47% | 74.89% | 92.66% | 87.37% | 93.79% |
| 16 | Ward's algorithm | 75.76% | 72.99% | 84.09% | 68.52% | 94.89% | 80.25% |
| 17 | Ward's algorithm | 72.08% | 99.18% | 86.34% | 97.32% | 98.64% | 80.80% |
| 18 | Ward's algorithm | 77.12% | 87.41% | 73.86% | 86.92% | 89.63% | 93.08% |
| 19 | Ward's algorithm | 81.85% | 82.35% | 86.46% | 76.78% | 97.83% | 86.34% |
| 20 | Correlation K-means | 82.64% | 82.64% | 89.81% | 80.13% | 96.98% | 86.53% |
| 21 | Ward's algorithm | 94.85% | 73.21% | 97.25% | 74.08% | 95.49% | 94.24% |
| 22 | Ward's algorithm | 79.78% | 97.93% | 77.53% | 99.28% | 93.45% | 97.48% |
| 23 | Ward's algorithm | 80.45% | 94.72% | 86.86% | 93.13% | 96.60% | 87.97% |
| 24 | Ward's algorithm | 95.28% | 71.18% | 96.85% | 66.49% | 94.12% | 86.49% |
| 26 | Cityblock K-means | 88.76% | 78.22% | 88.76% | 80.34% | 92.41% | 94.92% |
| 28 | Ward's algorithm | 35.86% | 85.25% | 34.48% | 90.91% | 88.52% | 98.18% |
| 30 | Correlation K-means | 79.02% | 88.93% | 89.51% | 88.35% | 98.52% | 86.41% |
| 33 | Correlation K-means | 84.85% | 96.46% | 86.72% | 92.89% | 96.70% | 91.11% |
| 34 | Correlation K-means | 39.86% | 85.51% | 45.27% | 80.72% | 98.55% | 81.93% |
| 35 | Correlation K-means | 81.23% | 96.50% | 80.70% | 92.90% | 96.82% | 93.83% |
| 37 | Cityblock K-means | 86.17% | 85.71% | 94.15% | 77.97% | 98.94% | 82.38% |
| 38 | Ward's algorithm | 39.20% | 89.91% | 55.20% | 90.20% | 98.17% | 69.93% |
| 39 | Cityblock K-means | 79.90% | 93.73% | 90.33% | 87.65% | 98.81% | 81.73% |
| 41 | Cityblock K-means | 81.96% | 76.08% | 77.32% | 72.12% | 90.91% | 91.35% |
| 42 | Cityblock K-means | 82.25% | 98.70% | 94.81% | 96.48% | 98.70% | 83.70% |
| 43 | Correlation K-means | 41.96% | 90.91% | 53.15% | 89.41% | 100.00% | 77.65% |
| 45 | Correlation K-means | 73.43% | 93.75% | 73.43% | 92.92% | 94.64% | 93.81% |
| 53 | Correlation K-means | 23.23% | 85.19% | 36.36% | 83.72% | 96.30% | 60.47% |
| 55 | Correlation K-means | 69.79% | 74.44% | 80.21% | 79.38% | 86.67% | 80.41% |
| 58 | Correlation K-means | 51.06% | 52.17% | 60.64% | 67.06% | 75.00% | 81.18% |
| 59 | Cityblock K-means | 68.10% | 64.75% | 69.83% | 62.79% | 86.89% | 82.17% |
| 60 | Cityblock K-means | 73.71% | 86.14% | 79.90% | 71.43% | 95.18% | 72.81% |
| 61 | Correlation K-means | 88.05% | 80.00% | 89.31% | 78.45% | 96.00% | 92.82% |
| 63 | Correlation K-means | 80.77% | 84.31% | 84.97% | 78.39% | 97.45% | 86.13% |
| 64 | Cityblock K-means | 65.55% | 71.73% | 75.60% | 61.48% | 94.76% | 70.43% |
| 69 | Cityblock K-means | 41.51% | 70.97% | 43.40% | 74.19% | 93.55% | 93.55% |
| 71 | Ward's algorithm | 36.12% | 93.14% | 44.11% | 69.05% | 84.31% | 51.19% |
| 72 | Cityblock K-means | 85.16% | 90.94% | 80.57% | 91.57% | 91.70% | 97.59% |
| 73 | Ward's algorithm | 61.61% | 90.05% | 78.33% | 88.15% | 99.10% | 76.31% |
| 74 | Ward's algorithm | 91.27% | 86.86% | 89.58% | 87.60% | 93.57% | 96.14% |
| 75 | Correlation K-means | 84.06% | 84.74% | 83.27% | 92.07% | 88.76% | 97.36% |
| 77 | Correlation K-means | 48.84% | 77.78% | 50.39% | 83.33% | 90.12% | 93.59% |
| 78 | Correlation K-means | 82.46% | 94.44% | 85.45% | 93.47% | 98.72% | 94.29% |
| 79 | Ward's algorithm | 43.36% | 84.48% | 61.06% | 81.18% | 98.28% | 67.06% |
| 80 | Ward's algorithm | 61.84% | 78.99% | 58.55% | 78.07% | 89.08% | 92.98% |
| 81 | Ward's algorithm | 93.49% | 84.41% | 90.55% | 91.75% | 88.82% | 99.67% |
| 83 | Ward's algorithm | 93.75% | 92.51% | 96.88% | 91.95% | 98.24% | 94.49% |
| 84 | Correlation K-means | 66.67% | 90.00% | 70.99% | 92.00% | 95.00% | 91.20% |
| 87 | Ward's algorithm | 64.88% | 91.81% | 61.98% | 92.02% | 88.30% | 92.64% |
| 89 | Correlation K-means | 82.57% | 97.43% | 84.53% | 98.73% | 96.66% | 95.67% |
| 90 | Correlation K-means | 59.62% | 52.54% | 44.23% | 56.10% | 62.71% | 90.24% |
| 91 | Cityblock K-means | 63.27% | 92.54% | 62.76% | 89.13% | 91.79% | 89.13% |
| 94 | Cityblock K-means | 81.77% | 84.86% | 80.73% | 85.64% | 93.51% | 95.58% |
| 96 | Cityblock K-means | 82.88% | 81.76% | 85.62% | 80.65% | 97.30% | 92.90% |
| 97 | Correlation K-means | 96.71% | 73.87% | 95.39% | 79.67% | 86.43% | 94.51% |
| 99 | Cityblock K-means | 72.46% | 79.37% | 71.01% | 85.96% | 88.89% | 98.25% |
| 100 | Cityblock K-means | 76.25% | 79.22% | 93.75% | 55.56% | 98.70% | 56.30% |
| Average | | 73.04% | 84.03% | 76.48% | 82.48% | 92.81% | 86.68% |
| Stdev | | 17.72% | 10.85% | 16.99% | 11.53% | 7.39% | 11.07% |

TABLE 3
Stage N3 classification accuracy for the 10 healthy patients

| | | Scorer 1 | | Scorer 2 | | Both scorers | |
|---|---|---|---|---|---|---|---|
| Volunteer | Algorithm | Precision | Sensitivity | Precision | Sensitivity | Precision | Sensitivity |
| C1 | Ward's algorithm | 79.88% | 83.33% | 71.60% | 84.03% | 83.95% | 94.44% |
| C2 | Correlation | 66.13% | 82.00% | 55.65% | 94.52% | 72.00% | 98.63% |
| C3 | Correlation | 59.22% | 76.25% | 55.34% | 78.08% | 83.75% | 91.78% |
| C4 | Correlation | 84.47% | 94.44% | 75.78% | 96.06% | 86.11% | 97.64% |
| C5 | Correlation | 88.63% | 88.04% | 84.95% | 89.75% | 91.36% | 97.17% |
| C6 | Correlation | 52.63% | 87.72% | 49.47% | 90.38% | 84.21% | 92.31% |
| C7 | Correlation | 82.43% | 88.83% | 76.13% | 91.85% | 88.83% | 99.46% |
| C8 | Ward's algorithm | 96.01% | 96.27% | 96.01% | 96.27% | 100.00% | 100.00% |
| C9 | Correlation | 53.45% | 76.23% | 53.45% | 76.23% | 100.00% | 100.00% |
| C10 | Correlation | 50.21% | 92.91% | 50.21% | 92.91% | 100.00% | 100.00% |
| Average | | 71.31% | 86.60% | 66.86% | 89.01% | 89.02% | 97.14% |
| Stdev | | 16.89% | 7.07% | 16.27% | 7.20% | 9.08% | 3.19% |

N2 Detection: Table 4 shows the accuracy of classifying REM stages for the 61 patients in the adult population with OSA, along with the scorers' agreements accuracy. Table 5 shows the same value for the 10 healthy individuals. A sleep stage smoothing algorithm was applied. Across all healthy individuals, the algorithm achieved a precision of 62.3±8.0% against scorer 1 and 67.3±9.1% against scorer 2; the algorithm achieved a sensitivity (recall) of 73.2±17.5% against scorer 1 and 73.7±17.6% against scorer 2.

TABLE 4
Stage N2 classification accuracy for the 61 adult patients with OSA.

| | Scorer 1 | | Scorer 2 | | Both scorers | |
|---|---|---|---|---|---|---|
| Patient | Precision | Sensitivity | Precision | Sensitivity | Precision | Sensitivity |
| 1 | 75.31% | 70.52% | 85.80% | 72.77% | 80.35% | 72.77% |
| 2 | 72.54% | 62.21% | 86.10% | 59.76% | 94.77% | 76.71% |
| 5 | 69.70% | 69.43% | 81.06% | 68.59% | 95.09% | 80.77% |
| 7 | 42.72% | 85.85% | 56.55% | 84.73% | 83.90% | 62.55% |
| 8 | 77.88% | 62.63% | 65.49% | 60.41% | 65.12% | 74.69% |
| 9 | 54.20% | 90.91% | 53.15% | 85.15% | 73.31% | 70.03% |
| 10 | 65.47% | 47.40% | 49.33% | 36.67% | 84.09% | 86.33% |
| 11 | 79.47% | 55.11% | 20.00% | 22.75% | 45.99% | 75.45% |
| 15 | 48.97% | 61.69% | 72.16% | 58.09% | 87.01% | 55.60% |
| 16 | 69.39% | 72.34% | 71.43% | 77.04% | 76.60% | 79.25% |
| 17 | 45.45% | 4.98% | 59.09% | 7.10% | 68.66% | 75.41% |
| 18 | 83.78% | 76.64% | 88.83% | 72.61% | 95.13% | 85.00% |
| 19 | 58.60% | 57.07% | 70.43% | 52.61% | 86.39% | 66.27% |
| 20 | 44.27% | 74.17% | 62.45% | 71.49% | 83.44% | 57.01% |
| 21 | 45.56% | 65.70% | 42.34% | 67.31% | 65.70% | 72.44% |
| 22 | 72.20% | 58.39% | 58.09% | 73.68% | 53.02% | 83.16% |
| 23 | 64.44% | 73.10% | 59.27% | 68.42% | 77.93% | 79.30% |
| 24 | 67.00% | 83.20% | 70.30% | 73.96% | 89.34% | 75.69% |
| 26 | 50.94% | 64.90% | 60.75% | 60.30% | 83.65% | 65.17% |
| 28 | 55.46% | 37.93% | 37.82% | 36.29% | 47.70% | 66.94% |
| 30 | 75.93% | 75.46% | 81.48% | 75.86% | 90.49% | 84.77% |
| 33 | 83.46% | 81.27% | 70.77% | 79.31% | 76.40% | 87.93% |
| 34 | 64.59% | 70.59% | 59.49% | 75.00% | 73.07% | 84.29% |
| 35 | 72.89% | 69.94% | 76.51% | 67.20% | 94.80% | 86.77% |
| 37 | 63.19% | 77.60% | 54.40% | 83.50% | 74.40% | 93.00% |
| 38 | 68.52% | 68.52% | 61.00% | 75.52% | 71.03% | 87.93% |
| 39 | 92.55% | 46.77% | 72.34% | 45.03% | 75.81% | 93.38% |
| 41 | 55.04% | 45.95% | 52.71% | 52.92% | 72.49% | 87.16% |
| 42 | 66.99% | 65.12% | 62.20% | 67.36% | 77.21% | 86.01% |
| 43 | 51.30% | 69.80% | 66.57% | 71.96% | 76.47% | 60.75% |
| 45 | 65.01% | 91.67% | 73.52% | 86.15% | 90.00% | 74.79% |
| 53 | 68.69% | 81.77% | 69.59% | 80.89% | 86.33% | 84.29% |
| 55 | 75.85% | 85.44% | 57.32% | 93.63% | 64.56% | 93.63% |
| 58 | 73.55% | 81.43% | 87.31% | 76.46% | 95.95% | 75.89% |
| 59 | 45.32% | 71.43% | 65.50% | 68.50% | 83.41% | 55.35% |
| 60 | 58.58% | 61.99% | 59.22% | 63.54% | 69.52% | 70.49% |
| 61 | 60.49% | 79.03% | 64.51% | 73.33% | 82.26% | 71.58% |
| 63 | 74.42% | 60.57% | 53.10% | 60.35% | 61.83% | 86.34% |
| 64 | 44.94% | 64.91% | 34.01% | 65.63% | 56.73% | 75.78% |
| 69 | 59.35% | 88.04% | 65.48% | 80.56% | 90.43% | 75.00% |
| 71 | 64.53% | 51.34% | 39.19% | 53.21% | 47.31% | 80.73% |
| 72 | 62.50% | 52.04% | 59.24% | 47.19% | 85.07% | 81.39% |
| 73 | 67.64% | 84.72% | 61.27% | 82.21% | 80.07% | 85.77% |
| 74 | 66.45% | 76.01% | 63.23% | 72.59% | 79.34% | 79.63% |
| 75 | 61.32% | 59.09% | 75.00% | 63.10% | 78.64% | 68.65% |
| 77 | 70.53% | 67.77% | 74.92% | 53.35% | 89.16% | 66.07% |
| 78 | 70.87% | 57.94% | 77.67% | 70.80% | 72.62% | 80.97% |
| 79 | 80.95% | 74.73% | 83.04% | 74.80% | 90.66% | 88.47% |
| 80 | 77.85% | 73.43% | 87.03% | 61.38% | 93.43% | 69.87% |
| 81 | 74.86% | 64.42% | 84.36% | 52.80% | 94.71% | 68.88% |
| 83 | 91.06% | 64.00% | 94.31% | 52.13% | 95.71% | 75.28% |
| 84 | 60.93% | 71.58% | 64.43% | 70.16% | 83.90% | 77.78% |
| 87 | 89.16% | 43.53% | 92.77% | 47.53% | 82.06% | 86.11% |
| 89 | 67.94% | 73.03% | 51.92% | 72.33% | 63.67% | 82.52% |
| 90 | 73.71% | 93.93% | 70.19% | 89.92% | 79.68% | 80.11% |
| 91 | 73.90% | 83.38% | 80.36% | 77.94% | 93.00% | 79.95% |
| 94 | 82.54% | 90.58% | 79.59% | 89.37% | 92.21% | 94.35% |
| 96 | 80.95% | 75.08% | 76.19% | 79.43% | 77.92% | 87.59% |
| 97 | 49.40% | 96.70% | 55.42% | 92.00% | 87.74% | 74.40% |
| 99 | 70.89% | 87.67% | 65.77% | 91.04% | 88.33% | 98.88% |
| 100 | 67.44% | 91.25% | 68.82% | 85.63% | 89.06% | 81.90% |
| Average | 66.71% | 69.57% | 66.27% | 67.73% | 79.42% | 78.11% |
| Stdev | 12.15% | 15.84% | 14.72% | 16.61% | 12.64% | 9.73% |

TABLE 5
Stage N2 classification accuracy for the 10 healthy individual

| | Scorer 1 | | Scorer 2 | | Both scorers | |
|---|---|---|---|---|---|---|
| Volunteer | Precision | Sensitivity | Precision | Sensitivity | Precision | Sensitivity |
| C1 | 69.20% | 88.89% | 72.15% | 89.30% | 92.68% | 89.30% |
| C2 | 67.18% | 81.73% | 69.47% | 84.78% | 80.19% | 80.43% |
| C3 | 68.52% | 44.58% | 80.25% | 49.62% | 85.14% | 80.92% |
| C4 | 67.55% | 87.18% | 65.56% | 90.83% | 76.50% | 82.11% |
| C5 | 64.86% | 80.90% | 72.97% | 84.97% | 85.39% | 79.72% |
| C6 | 69.42% | 66.55% | 78.78% | 65.96% | 87.24% | 76.20% |
| C7 | 46.12% | 76.77% | 63.57% | 66.13% | 92.26% | 57.66% |
| C8 | 58.86% | 95.88% | 58.86% | 95.88% | 100.00% | 100.00% |
| C9 | 58.73% | 61.33% | 58.73% | 61.33% | 100.00% | 100.00% |
| C10 | 52.53% | 47.98% | 52.53% | 47.98% | 100.00% | 100.00% |
| Average | 62.30% | 73.18% | 67.29% | 73.68% | 89.94% | 84.63% |
| Stdev | 8.00% | 17.45% | 9.06% | 17.61% | 8.46% | 13.29% |

N3 Detection: Table 6 shows the accuracy of classifying N3 stages for the 61 patients in the adult population with OSA, along with the scorers' agreements accuracy. Table 7 shows similar value for the 10 healthy individuals. A sleep stage smoothing algorithm was applied. Across all patients with OSA, the algorithm achieved a precision of 77.0±20.9% against scorer 1 and 76.5±21.4% against scorer 2; the algorithm achieved a sensitivity (recall) of 77.4±21.0% against scorer 1 and 79.2±22.4% against scorer 2. Across all healthy individuals, the algorithm achieved a precision of 87.3±8.0% against scorer 1 and 88.1±8.9% against scorer 2; the algorithm achieved a sensitivity (recall) of 78.6±14.7% against scorer 1 and 91.2+/−8.5% against scorer 2.

TABLE 6
Stage N3 classification accuracy for the 61 adult patients with OSA.

| | Scorer 1 | | Scorer 2 | | Both scorers | |
|---|---|---|---|---|---|---|
| Patient | Precision | Sensitivity | Precision | Sensitivity | Precision | Sensitivity |
| 1 | 93.12% | 87.88% | 98.62% | 93.07% | 89.61% | 89.61% |
| 2 | 70.74% | 84.71% | 53.72% | 94.39% | 66.88% | 98.13% |
| 5 | 94.51% | 94.51% | 96.95% | 96.36% | 97.56% | 96.97% |
| 7 | 87.80% | 61.54% | 94.51% | 66.24% | 86.32% | 86.32% |
| 8 | 76.77% | 91.12% | 91.73% | 76.64% | 97.20% | 68.42% |
| 9 | 98.46% | 40.25% | 100.00% | 29.55% | 96.23% | 69.55% |
| 10 | 63.16% | 25.00% | 76.32% | 20.57% | 96.88% | 65.96% |
| 11 | 84.42% | 93.85% | 77.89% | 99.36% | 86.59% | 99.36% |
| 15 | 87.67% | 70.72% | 93.84% | 80.12% | 86.19% | 91.23% |
| 16 | 64.80% | 96.67% | 85.47% | 83.15% | 99.17% | 64.67% |
| 17 | 36.99% | 98.18% | 46.58% | 100.00% | 85.45% | 69.12% |
| 18 | 70.73% | 75.32% | 64.63% | 82.81% | 76.62% | 92.19% |
| 19 | 73.49% | 89.71% | 63.86% | 96.36% | 75.00% | 92.73% |
| 20 | 96.10% | 71.84% | 93.07% | 80.52% | 84.79% | 98.13% |
| 21 | 42.55% | 100.00% | 46.10% | 89.66% | 97.50% | 80.69% |
| 22 | 55.06% | 45.79% | 74.16% | 31.58% | 100.00% | 51.20% |
| 23 | 27.91% | 18.46% | 51.16% | 17.74% | 100.00% | 52.42% |
| 24 | 81.41% | 94.78% | 83.33% | 100.00% | 90.30% | 93.08% |
| 26 | 94.04% | 86.06% | 88.08% | 100.00% | 80.61% | 100.00% |
| 28 | 70.37% | 96.31% | 90.24% | 79.53% | 100.00% | 64.39% |
| 30 | 54.55% | 100.00% | 50.00% | 100.00% | 83.33% | 90.91% |
| 33 | 94.32% | 92.22% | 85.23% | 96.15% | 86.67% | 100.00% |
| 34 | 96.55% | 64.00% | 96.55% | 66.67% | 89.71% | 93.45% |
| 35 | 86.71% | 99.28% | 88.61% | 98.59% | 98.55% | 95.77% |
| 37 | 68.85% | 85.71% | 87.70% | 79.85% | 100.00% | 73.13% |
| 38 | 40.22% | 97.37% | 55.43% | 99.03% | 98.68% | 72.82% |
| 39 | 89.86% | 95.38% | 94.93% | 84.52% | 98.46% | 82.58% |
| 41 | 64.10% | 87.72% | 68.91% | 89.96% | 92.98% | 88.70% |
| 42 | 1.75% | 2.38% | 14.04% | 12.31% | 76.19% | 49.23% |
| 43 | 76.92% | 55.56% | 74.04% | 70.64% | 59.72% | 78.90% |
| 45 | 96.71% | 76.17% | 91.45% | 89.68% | 78.24% | 97.42% |
| 53 | 86.71% | 58.37% | 82.66% | 68.42% | 78.99% | 97.13% |
| 55 | 91.02% | 76.38% | 100.00% | 61.17% | 100.00% | 72.89% |
| 58 | 78.38% | 66.41% | 75.68% | 76.36% | 77.86% | 92.73% |
| 59 | 83.26% | 66.43% | 65.16% | 84.71% | 61.01% | 99.41% |
| 60 | 65.99% | 84.12% | 68.69% | 90.67% | 78.97% | 81.78% |
| 61 | 92.39% | 76.92% | 78.80% | 89.51% | 73.30% | 100.00% |
| 63 | 83.70% | 76.24% | 92.39% | 57.43% | 97.03% | 66.22% |
| 64 | 90.22% | 92.74% | 86.41% | 98.15% | 89.39% | 98.77% |
| 69 | 98.97% | 65.53% | 94.33% | 66.06% | 88.40% | 93.50% |
| 71 | 38.52% | 58.02% | 40.98% | 51.55% | 86.42% | 72.16% |
| 72 | 56.78% | 99.26% | 55.93% | 98.51% | 90.37% | 91.04% |
| 73 | 100.00% | 50.30% | 96.43% | 47.65% | 92.81% | 91.18% |
| 74 | 45.71% | 76.19% | 43.81% | 100.00% | 65.08% | 89.13% |
| 75 | 100.00% | 63.10% | 93.40% | 80.49% | 73.21% | 100.00% |
| 77 | 65.93% | 93.13% | 21.68% | 90.74% | 33.75% | 100.00% |
| 78 | 85.49% | 80.49% | 90.67% | 82.55% | 86.34% | 83.49% |
| 79 | 79.62% | 93.28% | 85.35% | 92.41% | 92.54% | 85.52% |
| 80 | 75.91% | 93.30% | 51.82% | 100.00% | 63.13% | 99.12% |
| 81 | 80.18% | 97.33% | 59.03% | 99.26% | 71.12% | 98.52% |
| 83 | 62.01% | 96.52% | 32.96% | 98.33% | 47.83% | 91.67% |
| 84 | 93.36% | 69.28% | 92.19% | 71.30% | 87.54% | 91.24% |
| 87 | 55.16% | 93.37% | 51.25% | 98.63% | 83.73% | 95.21% |
| 89 | 90.83% | 63.37% | 100.00% | 48.78% | 98.26% | 68.70% |
| 90 | 100.00% | 82.23% | 100.00% | 92.05% | 88.83% | 99.43% |
| 91 | 78.95% | 76.27% | 68.42% | 95.90% | 68.93% | 100.00% |
| 94 | 90.15% | 92.25% | 90.15% | 94.44% | 96.90% | 99.21% |
| 96 | 97.18% | 86.43% | 100.00% | 74.68% | 100.00% | 83.97% |
| 97 | 98.86% | 66.67% | 93.18% | 71.30% | 86.59% | 98.26% |
| 99 | 100.00% | 58.08% | 100.00% | 55.42% | 99.56% | 95.00% |
| 100 | 88.04% | 93.64% | 78.26% | 91.14% | 83.24% | 91.14% |
| Average | 76.95% | 77.44% | 76.50% | 79.22% | 85.19% | 86.45% |
| Stdev | 20.88% | 20.97% | 21.36% | 22.39% | 13.81% | 13.75% |

TABLE 7
Stage N3 classification accuracy for the 10 healthy individuals.

| | Scorer 1 | | Scorer 2 | | Both scorers | |
|---|---|---|---|---|---|---|
| Volunteer | Precision | Sensitivity | Precision | Sensitivity | Precision | Sensitivity |
| C1 | 97.96% | 53.93% | 100.00% | 51.58% | 94.38% | 88.42% |
| C2 | 82.24% | 89.34% | 88.32% | 77.78% | 95.94% | 77.78% |
| C3 | 81.10% | 89.26% | 79.88% | 97.40% | 88.59% | 98.14% |
| C4 | 89.58% | 81.13% | 95.83% | 87.34% | 87.42% | 87.97% |
| C5 | 89.40% | 69.23% | 83.44% | 86.90% | 73.85% | 99.31% |
| C6 | 77.22% | 87.85% | 77.22% | 91.95% | 86.64% | 90.68% |
| C7 | 96.88% | 59.16% | 97.50% | 66.10% | 85.11% | 94.49% |
| C8 | 96.12% | 69.23% | 96.12% | 69.23% | 100.00% | 100.00% |
| C9 | 86.31% | 92.44% | 86.31% | 92.44% | 100.00% | 100.00% |
| C10 | 76.26% | 94.64% | 76.26% | 94.64% | 100.00% | 100.00% |
| Average | 87.31% | 78.62% | 88.09% | 81.54% | 91.19% | 93.68% |
| Stdev | 8.04% | 14.65% | 8.85% | 14.96% | 8.47% | 7.41% |

REM Detection: Table 8 shows the accuracy of classifying REM stages for the 61 patients in the adult population with OSA, along with the scorers' agreements accuracy. Table 9 shows the same value for the 10 healthy individuals. A sleep stage smoothing algorithm was applied. Across all patients with OSA, the algorithm achieved a precision of 66.7±12.2% against scorer 1 and 66.3±14.7% against scorer 2; the algorithm achieved a sensitivity (recall) of 69.6±15.8% against scorer 1 and 79.4±12.6% against scorer 2. Across all healthy individuals, the algorithm achieved a precision of 60.2±16.7% against scorer 1 and 67.3±17.4% against scorer 2; the algorithm achieved a sensitivity (recall) of 85.0±5.6% against scorer 1 and 84.8±6.7% against scorer 2.

TABLE 8
Stage REM classification accuracy for the 61 adult patients with OSA.

| | Scorer 1 | | Scorer 2 | | Both scorers | |
|---|---|---|---|---|---|---|
| Patient | Precision | Sensitivity | Precision | Sensitivity | Precision | Sensitivity |
| 1 | 75.31% | 70.52% | 85.80% | 72.77% | 80.35% | 72.77% |
| 2 | 72.54% | 62.21% | 86.10% | 59.76% | 94.77% | 76.71% |
| 5 | 69.70% | 69.43% | 81.06% | 68.59% | 95.09% | 80.77% |
| 7 | 42.72% | 85.85% | 56.55% | 84.73% | 83.90% | 62.55% |
| 8 | 77.88% | 62.63% | 65.49% | 60.41% | 65.12% | 74.69% |
| 9 | 54.20% | 90.91% | 53.15% | 85.15% | 73.31% | 70.03% |
| 10 | 65.47% | 47.40% | 49.33% | 36.67% | 84.09% | 86.33% |
| 11 | 79.47% | 55.11% | 20.00% | 22.75% | 45.99% | 75.45% |
| 15 | 48.97% | 61.69% | 72.16% | 58.09% | 87.01% | 55.60% |
| 16 | 69.39% | 72.34% | 71.43% | 77.04% | 76.60% | 79.25% |
| 17 | 45.45% | 4.98% | 59.09% | 7.10% | 68.66% | 75.41% |
| 18 | 83.78% | 76.64% | 88.83% | 72.61% | 95.13% | 85.00% |
| 19 | 58.60% | 57.07% | 70.43% | 52.61% | 86.39% | 66.27% |
| 20 | 44.27% | 74.17% | 62.45% | 71.49% | 83.44% | 57.01% |
| 21 | 45.56% | 65.70% | 42.34% | 67.31% | 65.70% | 72.44% |
| 22 | 72.20% | 58.39% | 58.09% | 73.68% | 53.02% | 83.16% |
| 23 | 64.44% | 73.10% | 59.27% | 68.42% | 77.93% | 79.30% |
| 24 | 67.00% | 83.20% | 70.30% | 73.96% | 89.34% | 75.69% |
| 26 | 50.94% | 64.90% | 60.75% | 60.30% | 83.65% | 65.17% |
| 28 | 55.46% | 37.93% | 37.82% | 36.29% | 47.70% | 66.94% |
| 30 | 75.93% | 75.46% | 81.48% | 75.86% | 90.49% | 84.77% |
| 33 | 83.46% | 81.27% | 70.77% | 79.31% | 76.40% | 87.93% |
| 34 | 64.59% | 70.59% | 59.49% | 75.00% | 73.07% | 84.29% |
| 35 | 72.89% | 69.94% | 76.51% | 67.20% | 94.80% | 86.77% |
| 37 | 63.19% | 77.60% | 54.40% | 83.50% | 74.40% | 93.00% |
| 38 | 68.52% | 68.52% | 61.00% | 75.52% | 71.03% | 87.93% |
| 39 | 92.55% | 46.77% | 72.34% | 45.03% | 75.81% | 93.38% |
| 41 | 55.04% | 45.95% | 52.71% | 52.92% | 72.49% | 87.16% |
| 42 | 66.99% | 65.12% | 62.20% | 67.36% | 77.21% | 86.01% |
| 43 | 51.30% | 69.80% | 66.57% | 71.96% | 76.47% | 60.75% |
| 45 | 65.01% | 91.67% | 73.52% | 86.15% | 90.00% | 74.79% |
| 53 | 68.69% | 81.77% | 69.59% | 80.89% | 86.33% | 84.29% |
| 55 | 75.85% | 85.44% | 57.32% | 93.63% | 64.56% | 93.63% |
| 58 | 73.55% | 81.43% | 87.31% | 76.46% | 95.95% | 75.89% |
| 59 | 45.32% | 71.43% | 65.50% | 68.50% | 83.41% | 55.35% |
| 60 | 58.58% | 61.99% | 59.22% | 63.54% | 69.52% | 70.49% |
| 61 | 60.49% | 79.03% | 64.51% | 73.33% | 82.26% | 71.58% |
| 63 | 74.42% | 60.57% | 53.10% | 60.35% | 61.83% | 86.34% |
| 64 | 44.94% | 64.91% | 34.01% | 65.63% | 56.73% | 75.78% |
| 69 | 59.35% | 88.04% | 65.48% | 80.56% | 90.43% | 75.00% |
| 71 | 64.53% | 51.34% | 39.19% | 53.21% | 47.31% | 80.73% |
| 72 | 62.50% | 52.04% | 59.24% | 47.19% | 85.07% | 81.39% |
| 73 | 67.64% | 84.72% | 61.27% | 82.21% | 80.07% | 85.77% |
| 74 | 66.45% | 76.01% | 63.23% | 72.59% | 79.34% | 79.63% |
| 75 | 61.32% | 59.09% | 75.00% | 63.10% | 78.64% | 68.65% |
| 77 | 70.53% | 67.77% | 74.92% | 53.35% | 89.16% | 66.07% |
| 78 | 70.87% | 57.94% | 77.67% | 70.80% | 72.62% | 80.97% |
| 79 | 80.95% | 74.73% | 83.04% | 74.80% | 90.66% | 88.47% |
| 80 | 77.85% | 73.43% | 87.03% | 61.38% | 93.43% | 69.87% |
| 81 | 74.86% | 64.42% | 84.36% | 52.80% | 94.71% | 68.88% |
| 83 | 91.06% | 64.00% | 94.31% | 52.13% | 95.71% | 75.28% |
| 84 | 60.93% | 71.58% | 64.43% | 70.16% | 83.90% | 77.78% |
| 87 | 89.16% | 43.53% | 92.77% | 47.53% | 82.06% | 86.11% |
| 89 | 67.94% | 73.03% | 51.92% | 72.33% | 63.67% | 82.52% |
| 90 | 73.71% | 93.93% | 70.19% | 89.92% | 79.68% | 80.11% |
| 91 | 73.90% | 83.38% | 80.36% | 77.94% | 93.00% | 79.95% |
| 94 | 82.54% | 90.58% | 79.59% | 89.37% | 92.21% | 94.35% |
| 96 | 80.95% | 75.08% | 76.19% | 79.43% | 77.92% | 87.59% |
| 97 | 49.40% | 96.70% | 55.42% | 92.00% | 87.74% | 74.40% |
| 99 | 70.89% | 87.67% | 65.77% | 91.04% | 88.33% | 98.88% |
| 100 | 67.44% | 91.25% | 68.82% | 85.63% | 89.06% | 81.90% |
| Average | 66.71% | 69.57% | 66.27% | 67.73% | 79.42% | 78.11% |
| Stdev | 12.15% | 15.84% | 14.72% | 16.61% | 12.64% | 9.73% |

TABLE 9
Stage REM classification accuracy for the 10 healthy individual

| | Scorer 1 | | Scorer 2 | | Both scorers | |
|---|---|---|---|---|---|---|
| Volunteer | Precision | Sensitivity | Precision | Sensitivity | Precision | Sensitivity |
| C1 | 54.75% | 97.03% | 55.31% | 97.06% | 93.07% | 92.16% |
| C2 | 73.89% | 88.67% | 80.00% | 86.23% | 93.33% | 83.83% |
| C3 | 82.35% | 81.55% | 85.29% | 73.73% | 97.09% | 84.75% |
| C4 | 50.00% | 84.44% | 69.74% | 83.46% | 94.44% | 66.93% |
| C5 | 50.81% | 77.78% | 62.90% | 80.41% | 96.30% | 80.41% |
| C6 | 55.26% | 87.50% | 81.58% | 90.51% | 96.88% | 67.88% |
| C7 | 65.15% | 87.76% | 68.18% | 90.91% | 87.76% | 86.87% |
| C8 | 75.90% | 84.56% | 75.90% | 84.56% | 100.00% | 100.00% |
| C9 | 25.13% | 79.37% | 25.13% | 79.37% | 100.00% | 100.00% |
| C10 | 69.18% | 81.48% | 69.18% | 81.48% | 100.00% | 100.00% |
| Average | 60.24% | 85.01% | 67.32% | 84.77% | 95.89% | 86.28% |
| Stdev | 16.69% | 5.58% | 17.37% | 6.72% | 3.89% | 12.27% |

Overall Accuracy: Table 10 shows the overall accuracy for all five stages for the 61 patients in the adult population with OSA, along with the scorers' agreements accuracy. Table 11 shows the same value for the 10 healthy individuals. Across all patients with OSA, the algorithm achieved an accuracy of 68.8±7.2% against scorer 1 and 69.5±7.4% against scorer 2. Across all healthy individuals, the algorithm achieved an accuracy of 69.0±6.0% against scorer 1 and 71.2±6.5% against scorer 2. FIGS. 8A-8B show the confidence matrices for the scorers against the algorithm for the 61 patients with OSA. The darker the values are, the higher the agreements are between the scorers and the algorithm. From the figure, the algorithm performed the best when classifying stages W, N3, and R. The algorithm performed the worst when classifying stages N1 and N2.

TABLE 10
All five stages classification accuracy
for the 61 adult patients with OSA.

| Patient | Scorer 1 | Scorer 2 | Both scorers |
|---|---|---|---|
| 1 | 83.29% | 86.47% | 87.29% |
| 2 | 69.27% | 67.02% | 83.40% |
| 5 | 74.56% | 79.17% | 88.40% |
| 7 | 56.70% | 63.79% | 81.40% |
| 8 | 69.68% | 68.88% | 76.54% |
| 9 | 60.06% | 58.89% | 71.57% |
| 10 | 59.24% | 54.68% | 84.85% |
| 11 | 72.18% | 57.29% | 71.77% |
| 15 | 67.99% | 72.35% | 78.84% |
| 16 | 66.94% | 72.68% | 77.61% |
| 17 | 56.15% | 67.48% | 75.52% |
| 18 | 74.72% | 75.85% | 87.31% |
| 19 | 66.29% | 66.79% | 80.95% |
| 20 | 66.74% | 71.30% | 82.17% |
| 21 | 58.98% | 59.57% | 86.23% |
| 22 | 63.49% | 60.68% | 75.46% |
| 23 | 66.82% | 66.82% | 79.81% |
| 24 | 72.13% | 72.63% | 83.25% |
| 26 | 70.25% | 72.00% | 81.49% |
| 28 | 59.62% | 62.44% | 78.29% |
| 30 | 72.65% | 78.40% | 84.51% |
| 33 | 85.28% | 81.91% | 86.52% |
| 34 | 60.52% | 62.19% | 79.90% |
| 35 | 77.57% | 77.04% | 90.24% |
| 37 | 67.65% | 68.94% | 84.28% |
| 38 | 56.10% | 60.42% | 75.28% |
| 39 | 69.66% | 72.41% | 83.56% |
| 41 | 60.59% | 59.32% | 82.20% |
| 42 | 71.10% | 77.75% | 80.95% |
| 43 | 56.07% | 64.02% | 70.29% |
| 45 | 74.09% | 78.31% | 83.22% |
| 53 | 69.55% | 70.45% | 83.07% |
| 55 | 75.27% | 68.43% | 78.45% |
| 58 | 62.57% | 71.44% | 79.57% |
| 59 | 57.79% | 61.07% | 74.12% |
| 60 | 64.56% | 66.25% | 73.93% |
| 61 | 68.00% | 68.25% | 80.78% |
| 63 | 68.94% | 67.10% | 76.84% |
| 64 | 65.78% | 63.69% | 77.73% |
| 69 | 76.43% | 74.65% | 86.50% |
| 71 | 53.69% | 47.56% | 62.58% |
| 72 | 71.02% | 67.93% | 87.89% |
| 73 | 63.29% | 65.15% | 80.46% |
| 74 | 74.05% | 71.28% | 82.93% |
| 75 | 70.05% | 75.12% | 80.99% |
| 77 | 64.26% | 56.77% | 69.55% |
| 78 | 73.38% | 76.85% | 83.10% |
| 79 | 69.65% | 74.00% | 86.24% |
| 80 | 65.77% | 67.52% | 74.04% |
| 81 | 78.21% | 75.60% | 83.88% |
| 83 | 77.99% | 71.17% | 83.58% |
| 84 | 70.85% | 72.24% | 85.64% |
| 87 | 68.08% | 67.63% | 85.16% |
| 89 | 76.74% | 75.94% | 82.11% |
| 90 | 71.21% | 70.99% | 77.08% |
| 91 | 73.65% | 73.54% | 86.04% |
| 94 | 81.86% | 82.23% | 91.30% |
| 96 | 75.85% | 75.85% | 84.66% |
| 97 | 70.63% | 72.56% | 83.79% |
| 99 | 78.14% | 76.52% | 94.29% |
| 100 | 73.65% | 73.77% | 80.99% |
| Average | 68.81% | 69.49% | 81.15% |
| Stdev | 7.19% | 7.37% | 5.68% |

TABLE 11
All five stages classification accuracy
for the 10 adult patients with OSA.

| | Scorer 1 | Scorer 2 | Both scorers |
|---|---|---|---|
| Volunteer | Sensitivity | Precision | Precision |
| C1 | 70.45% | 71.00% | 88.20% |
| C2 | 71.57% | 73.98% | 79.80% |
| C3 | 68.26% | 69.52% | 86.40% |
| C4 | 70.81% | 73.69% | 79.58% |
| C5 | 73.41% | 76.37% | 84.03% |
| C6 | 66.10% | 73.63% | 80.07% |
| C7 | 69.52% | 73.72% | 81.89% |
| C8 | 79.90% | 79.90% | 100.00% |
| C9 | 59.00% | 59.00% | 100.00% |
| C10 | 60.97% | 60.97% | 100.00% |
| Average | 69.00% | 71.18% | 88.00% |
| Stdev | 6.00% | 6.54% | 8.75% |

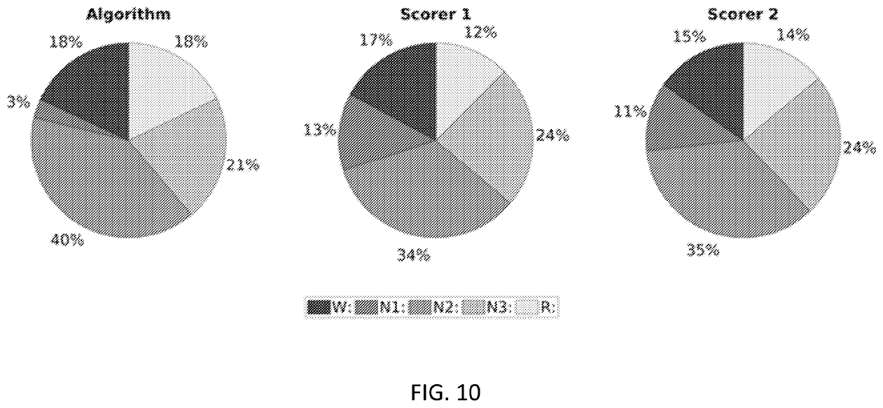

Sleep Stages, by Percentages: For clinical significance, one important factor is the percentage contribution of sleep stages to the overall sleep time. Table 12 shows the five-stage percentage contribution for the 61 patients in the adult population with OSA, along with the scorers' agreements accuracy. Table 13 shows the same value for the 10 healthy individuals. FIG. 10 shows the pie chart distribution of sleep stages, as graded by the professionals and the algorithm. From the figure, we can see that both the algorithm and the human scorers agree highly on the percentage of time spent in the W, N3, and R stages. The greatest discrepancy comes from the percentage of time spent in stage N1 and N2.

TABLE 12
Sleep stages contribution percentage of 61 Patients with OSA

| | Algorithm | | | | | Scorer | | | | | Scorer 2 | | | | |
|---|---|---|---|---|---|---|---|---|---|---|---|---|---|---|---|
| | | N1 | N2 | N3 | R | W | N1 | N2 | N3 | R | W | N1 | N2 | N3 | R |
| Patient | W | (%) | (%) | (%) | (%) | (%) | (%) | (%) | (%) | (%) | (%) | (%) | (%) | (%) | (%) |
| 1 | 33.18 | 9.18 | 19.06 | 25.65 | 12.94 | 30.35 | 8.24 | 20.35 | 27.18 | 13.88 | 31.29 | 4.59 | 22.47 | 27.18 | 14.47 |
| 2 | 29.82 | 5.38 | 27.13 | 26.63 | 11.04 | 27.86 | 9.70 | 28.98 | 21.75 | 11.72 | 26.51 | 8.02 | 34.53 | 18.95 | 12.00 |
| 5 | 30.47 | 5.36 | 28.45 | 24.31 | 11.41 | 29.02 | 10.69 | 29.75 | 21.00 | 9.55 | 28.72 | 7.00 | 35.30 | 19.13 | 9.85 |
| 7 | 25.25 | 6.54 | 32.84 | 22.73 | 12.63 | 25.40 | 12.80 | 27.94 | 22.25 | 11.61 | 25.37 | 8.07 | 34.06 | 20.87 | 11.64 |
| 8 | 24.90 | 7.88 | 31.46 | 23.99 | 11.78 | 24.74 | 13.07 | 28.78 | 22.70 | 10.71 | 25.03 | 7.56 | 32.86 | 23.63 | 10.92 |
| 9 | 24.04 | 6.66 | 36.63 | 20.99 | 11.67 | 22.66 | 13.99 | 30.10 | 21.68 | 11.56 | 22.88 | 7.91 | 33.77 | 23.59 | 11.84 |
| 10 | 25.71 | 7.91 | 35.42 | 18.84 | 12.12 | 24.38 | 13.61 | 31.14 | 20.38 | 10.49 | 24.35 | 7.63 | 34.19 | 22.77 | 11.06 |
| 11 | 26.53 | 8.10 | 33.28 | 19.08 | 13.01 | 25.72 | 13.11 | 30.76 | 20.13 | 10.29 | 27.29 | 8.63 | 31.89 | 21.87 | 10.32 |
| 15 | 26.76 | 8.34 | 32.55 | 19.10 | 13.25 | 25.66 | 12.88 | 29.76 | 20.49 | 11.21 | 26.92 | 8.40 | 31.89 | 21.94 | 10.85 |
| 16 | 25.66 | 8.39 | 33.30 | 19.28 | 13.36 | 24.72 | 13.37 | 30.62 | 19.87 | 11.42 | 26.14 | 8.51 | 32.42 | 21.91 | 11.03 |
| 17 | 28.74 | 8.50 | 30.67 | 19.15 | 12.94 | 26.44 | 13.78 | 30.09 | 18.74 | 10.95 | 28.58 | 8.65 | 31.55 | 20.73 | 10.49 |
| 18 | 27.55 | 8.39 | 31.42 | 18.95 | 13.69 | 25.29 | 13.52 | 31.23 | 18.47 | 11.49 | 27.19 | 8.59 | 33.01 | 20.04 | 11.17 |
| 19 | 28.48 | 9.08 | 30.85 | 18.35 | 13.25 | 26.36 | 13.89 | 30.71 | 17.77 | 11.26 | 28.50 | 8.59 | 32.89 | 19.11 | 10.91 |
| 20 | 28.50 | 8.88 | 30.59 | 18.85 | 13.17 | 26.54 | 13.64 | 29.64 | 18.96 | 11.22 | 28.79 | 8.42 | 32.22 | 19.86 | 10.71 |
| 21 | 28.50 | 9.05 | 30.10 | 19.53 | 12.83 | 27.34 | 13.61 | 28.65 | 18.40 | 12.00 | 29.45 | 8.65 | 30.91 | 19.42 | 11.57 |
| 22 | 29.37 | 8.58 | 30.06 | 19.02 | 12.97 | 27.80 | 13.22 | 29.10 | 18.09 | 11.79 | 29.71 | 8.66 | 30.46 | 19.77 | 11.40 |
| 23 | 29.76 | 8.52 | 30.53 | 18.21 | 12.98 | 27.97 | 13.19 | 29.36 | 17.48 | 11.99 | 29.94 | 8.46 | 30.61 | 19.46 | 11.53 |
| 24 | 29.05 | 8.74 | 30.90 | 18.28 | 13.03 | 27.63 | 13.06 | 29.42 | 17.45 | 12.44 | 29.59 | 8.31 | 30.88 | 19.30 | 11.91 |
| 26 | 28.86 | 9.41 | 30.58 | 18.05 | 13.09 | 27.74 | 13.53 | 28.85 | 17.36 | 12.53 | 29.53 | 8.93 | 30.58 | 18.91 | 12.06 |
| 28 | 28.29 | 9.61 | 29.78 | 18.87 | 13.45 | 26.74 | 13.41 | 28.44 | 17.75 | 13.66 | 28.42 | 8.57 | 29.80 | 19.90 | 13.31 |
| 30 | 28.63 | 9.29 | 30.16 | 18.71 | 13.21 | 26.98 | 13.45 | 28.89 | 17.32 | 13.36 | 28.78 | 8.48 | 30.31 | 19.34 | 13.08 |
| 33 | 29.81 | 8.94 | 30.12 | 18.30 | 12.84 | 27.93 | 13.11 | 28.94 | 16.99 | 13.03 | 29.78 | 8.48 | 30.12 | 18.86 | 12.77 |
| 34 | 29.30 | 8.59 | 30.61 | 18.12 | 13.38 | 27.11 | 13.30 | 29.34 | 17.15 | 13.11 | 28.95 | 8.91 | 30.25 | 18.90 | 12.99 |
| 35 | 30.02 | 8.36 | 30.29 | 18.22 | 13.11 | 27.62 | 13.24 | 29.10 | 17.19 | 12.85 | 29.45 | 8.91 | 30.06 | 18.90 | 12.69 |
| 37 | 29.81 | 8.17 | 30.62 | 18.13 | 13.27 | 27.51 | 13.32 | 29.21 | 17.02 | 12.94 | 29.44 | 9.02 | 29.90 | 18.84 | 12.79 |
| 38 | 29.73 | 7.99 | 30.99 | 18.22 | 13.07 | 26.89 | 13.73 | 29.63 | 16.68 | 13.06 | 28.95 | 9.77 | 29.99 | 18.54 | 12.75 |
| 39 | 30.30 | 8.33 | 30.24 | 18.13 | 13.00 | 27.32 | 13.80 | 29.33 | 16.62 | 12.93 | 29.60 | 9.82 | 29.53 | 18.52 | 12.54 |
| 41 | 29.92 | 8.18 | 30.13 | 18.71 | 13.06 | 27.12 | 13.60 | 29.46 | 16.91 | 12.91 | 29.31 | 9.99 | 29.44 | 18.78 | 12.49 |
| 42 | 30.83 | 7.99 | 30.03 | 18.35 | 12.81 | 27.81 | 13.58 | 29.40 | 16.55 | 12.66 | 30.20 | 9.80 | 29.29 | 18.46 | 12.26 |
| 43 | 30.53 | 7.89 | 30.53 | 18.25 | 12.81 | 27.29 | 13.85 | 29.57 | 16.65 | 12.64 | 29.69 | 9.99 | 29.72 | 18.37 | 12.24 |
| 45 | 30.06 | 7.72 | 31.11 | 18.22 | 12.88 | 26.82 | 13.78 | 29.72 | 16.82 | 12.86 | 29.14 | 9.91 | 30.09 | 18.34 | 12.51 |
| 53 | 29.46 | 7.54 | 31.73 | 18.26 | 13.00 | 26.07 | 13.59 | 30.12 | 17.22 | 13.01 | 28.37 | 9.93 | 30.51 | 18.52 | 12.67 |
| 55 | 28.92 | 7.41 | 32.22 | 18.30 | 13.14 | 25.61 | 13.46 | 30.50 | 17.40 | 13.03 | 27.87 | 10.06 | 30.49 | 18.92 | 12.67 |
| 58 | 28.33 | 7.23 | 32.77 | 18.10 | 13.57 | 25.11 | 13.65 | 30.96 | 17.29 | 12.99 | 27.27 | 10.01 | 31.32 | 18.69 | 12.70 |
| 59 | 27.86 | 7.07 | 32.92 | 18.28 | 13.88 | 24.76 | 13.82 | 30.75 | 17.69 | 13.00 | 26.88 | 10.25 | 31.46 | 18.69 | 12.72 |
| 60 | 27.69 | 7.00 | 32.97 | 18.72 | 13.63 | 24.58 | 13.71 | 30.81 | 17.93 | 12.97 | 26.81 | 10.20 | 31.49 | 18.88 | 12.62 |
| 61 | 27.48 | 6.90 | 33.14 | 18.81 | 13.67 | 24.50 | 13.67 | 30.79 | 18.16 | 12.88 | 26.69 | 10.24 | 31.57 | 18.90 | 12.61 |
| 63 | 27.57 | 7.24 | 32.99 | 18.56 | 13.64 | 24.64 | 13.75 | 30.89 | 17.96 | 12.76 | 26.88 | 10.39 | 31.37 | 18.82 | 12.54 |
| 64 | 27.49 | 7.45 | 32.88 | 18.63 | 13.55 | 24.58 | 14.09 | 30.61 | 18.03 | 12.69 | 26.96 | 10.76 | 30.95 | 18.82 | 12.51 |
| 69 | 27.02 | 7.31 | 33.03 | 18.77 | 13.87 | 24.11 | 13.85 | 30.52 | 18.46 | 13.05 | 26.43 | 10.62 | 30.98 | 19.19 | 12.77 |
| 71 | 27.15 | 7.21 | 33.12 | 18.69 | 13.83 | 23.86 | 13.96 | 30.88 | 18.28 | 13.02 | 26.31 | 10.99 | 30.89 | 19.04 | 12.77 |
| 72 | 27.30 | 7.09 | 32.86 | 18.91 | 13.84 | 24.04 | 13.92 | 30.77 | 18.23 | 13.05 | 26.39 | 11.04 | 30.81 | 18.96 | 12.80 |
| 73 | 27.46 | 7.01 | 33.02 | 18.64 | 13.87 | 24.00 | 14.00 | 30.78 | 18.20 | 13.01 | 26.47 | 11.09 | 30.77 | 18.93 | 12.74 |
| 74 | 27.77 | 6.90 | 33.08 | 18.49 | 13.76 | 24.44 | 13.89 | 30.79 | 17.95 | 12.93 | 26.82 | 11.14 | 30.78 | 18.62 | 12.64 |
| 75 | 27.79 | 7.37 | 32.89 | 18.35 | 13.60 | 24.53 | 13.98 | 30.67 | 17.98 | 12.84 | 26.81 | 11.35 | 30.74 | 18.52 | 12.59 |
| 77 | 27.58 | 7.26 | 33.05 | 18.57 | 13.55 | 24.26 | 14.08 | 30.91 | 18.04 | 12.72 | 26.48 | 11.38 | 31.27 | 18.29 | 12.58 |
| 78 | 27.65 | 7.28 | 32.85 | 18.65 | 13.58 | 24.32 | 13.95 | 30.87 | 18.16 | 12.71 | 26.52 | 11.34 | 31.16 | 18.43 | 12.56 |
| 79 | 27.35 | 7.26 | 32.99 | 18.64 | 13.75 | 23.96 | 14.02 | 31.12 | 18.11 | 12.79 | 26.18 | 11.35 | 31.42 | 18.40 | 12.65 |
| 80 | 27.16 | 7.15 | 33.07 | 18.78 | 13.84 | 23.75 | 14.14 | 31.28 | 18.16 | 12.67 | 25.92 | 11.29 | 31.84 | 18.29 | 12.65 |
| 81 | 27.32 | 7.30 | 32.81 | 18.93 | 13.65 | 24.06 | 14.06 | 31.12 | 18.22 | 12.54 | 26.09 | 11.30 | 31.85 | 18.23 | 12.52 |
| 83 | 27.27 | 7.42 | 32.70 | 18.95 | 13.66 | 24.08 | 13.99 | 31.28 | 18.12 | 12.53 | 26.10 | 11.17 | 32.21 | 18.00 | 12.51 |
| 84 | 27.06 | 7.29 | 32.78 | 19.12 | 13.74 | 23.85 | 13.82 | 31.28 | 18.51 | 12.54 | 25.84 | 11.04 | 32.24 | 18.36 | 12.52 |
| 87 | 27.06 | 7.18 | 32.51 | 19.36 | 13.89 | 23.76 | 13.69 | 31.41 | 18.51 | 12.64 | 25.69 | 11.03 | 32.32 | 18.32 | 12.64 |
| 89 | 27.46 | 7.06 | 32.42 | 19.20 | 13.86 | 24.08 | 13.58 | 31.31 | 18.48 | 12.56 | 25.98 | 10.90 | 32.07 | 18.45 | 12.60 |
| 90 | 27.05 | 6.97 | 32.82 | 19.18 | 13.98 | 23.75 | 13.67 | 31.51 | 18.54 | 12.54 | 25.57 | 11.09 | 32.25 | 18.47 | 12.61 |
| 91 | 26.92 | 6.91 | 32.96 | 19.15 | 14.05 | 23.55 | 13.63 | 31.59 | 18.54 | 12.69 | 25.35 | 11.10 | 32.43 | 18.36 | 12.76 |
| 94 | 26.87 | 6.83 | 33.10 | 19.10 | 14.10 | 23.54 | 13.56 | 31.69 | 18.49 | 12.72 | 25.30 | 11.07 | 32.51 | 18.31 | 12.82 |
| 96 | 26.71 | 6.95 | 33.14 | 19.14 | 14.05 | 23.45 | 13.45 | 31.80 | 18.58 | 12.72 | 25.19 | 10.99 | 32.53 | 18.48 | 12.81 |
| 97 | 26.55 | 6.87 | 33.38 | 19.15 | 14.05 | 23.43 | 13.40 | 31.67 | 18.77 | 12.73 | 25.11 | 10.95 | 32.46 | 18.61 | 12.86 |
| 99 | 26.27 | 6.78 | 33.58 | 19.11 | 14.25 | 23.19 | 13.23 | 31.75 | 18.92 | 12.91 | 24.83 | 10.86 | 32.47 | 18.78 | 13.05 |
| 100 | 26.01 | 6.72 | 33.87 | 19.16 | 14.24 | 22.97 | 13.29 | 31.86 | 18.95 | 12.92 | 24.70 | 10.82 | 32.62 | 18.79 | 13.07 |
| Average | 28.03 | 7.67 | 31.77 | 19.23 | 13.30 | 25.50 | 13.41 | 30.14 | 18.52 | 12.43 | 27.30 | 9.69 | 31.36 | 19.38 | 12.27 |
| Stdev | 1.63 | 0.91 | 2.31 | 1.75 | 0.66 | 1.76 | 0.97 | 1.61 | 1.77 | 0.84 | 1.80 | 1.41 | 1.74 | 1.63 | 0.83 |

TABLE 13
Sleep stages contribution percentage of 10 healthy individuals

| | Algorithm | | | | | Scorer 1 | | | | | Scorer 2 | | | | |
|---|---|---|---|---|---|---|---|---|---|---|---|---|---|---|---|
| | W | N1 | N2 | N3 | R | W | N1 | N2 | N3 | R | W | N1 | N2 | N3 | R |
| Volunteer | (%) | (%) | (%) | (%) | (%) | (%) | (%) | (%) | (%) | (%) | (%) | (%) | (%) | (%) | (%) |
| C1 | 17.42 | 1.30 | 51.30 | 10.61 | 19.37 | 17.53 | 12.34 | 39.94 | 19.26 | 10.93 | 15.58 | 11.36 | 41.45 | 20.56 | 11.04 |
| C2 | 15.10 | 1.09 | 47.25 | 17.00 | 19.56 | 14.28 | 13.90 | 37.71 | 20.44 | 13.68 | 11.83 | 11.50 | 38.42 | 23.60 | 14.66 |
| C3 | 13.47 | 5.52 | 39.14 | 24.34 | 17.54 | 13.01 | 12.13 | 35.79 | 25.60 | 13.47 | 11.03 | 10.76 | 36.78 | 26.70 | 14.72 |
| C4 | 15.18 | 4.42 | 39.23 | 23.11 | 18.07 | 14.32 | 13.44 | 34.63 | 24.52 | 13.09 | 12.29 | 12.29 | 34.92 | 25.35 | 15.15 |
| C5 | 18.90 | 3.65 | 38.63 | 21.71 | 17.11 | 18.27 | 12.21 | 33.48 | 23.84 | 12.19 | 16.25 | 12.07 | 34.15 | 23.33 | 14.19 |
| C6 | 17.84 | 3.27 | 37.86 | 23.70 | 17.33 | 16.45 | 12.85 | 33.76 | 24.83 | 12.11 | 14.66 | 11.42 | 35.15 | 24.19 | 14.58 |
| C7 | 19.23 | 3.04 | 37.20 | 23.27 | 17.26 | 17.75 | 12.21 | 31.91 | 25.97 | 12.16 | 15.83 | 10.20 | 34.68 | 24.97 | 14.32 |
| C8 | 21.96 | 2.76 | 36.55 | 21.48 | 17.24 | 20.70 | 12.07 | 30.23 | 24.39 | 12.61 | 19.04 | 10.34 | 32.61 | 23.53 | 14.47 |
| C9 | 20.58 | 2.72 | 36.99 | 21.99 | 17.72 | 19.78 | 12.76 | 31.23 | 24.34 | 11.90 | 18.32 | 11.24 | 33.32 | 23.58 | 13.54 |
| C10 | 21.15 | 3.68 | 35.53 | 21.64 | 17.99 | 19.49 | 14.17 | 30.46 | 23.47 | 12.41 | 18.16 | 12.78 | 32.37 | 22.78 | 13.90 |
| Average | 18.08 | 3.15 | 39.97 | 20.89 | 17.92 | 17.16 | 12.81 | 33.91 | 23.67 | 12.45 | 15.30 | 11.40 | 35.39 | 23.86 | 14.06 |
| Stdev | 2.83 | 1.32 | 5.13 | 4.14 | 0.88 | 2.60 | 0.77 | 3.19 | 2.16 | 0.81 | 2.84 | 0.83 | 2.81 | 1.64 | 1.15 |

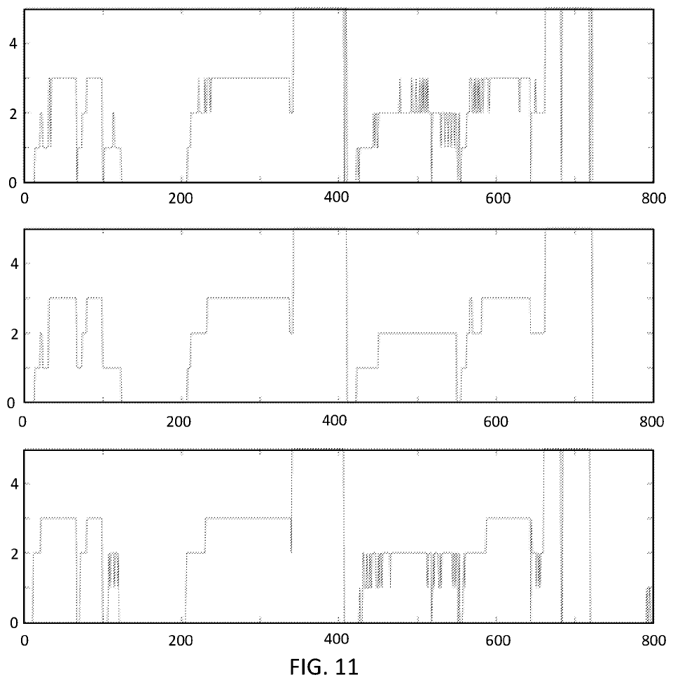

Sleep Stages, by Time: When grading sleep stages, professional use the knowledge of the previous sleep stages to infer on the current sleep stage (Berry et al., 2013). FIG. 11 compares the effect of smoothing sleep stages onto the algorithm's result. The middle panel shows the smoothed hypnogram; which is the final value of sleep stages that was used to compare against the scorers. While a throughout numerical evaluation was not available, visual inspection shows that applying smoothing onto the sleep stages produce results similar to that of the professionals. This is in line with the findings from Liang et al., which shows the improvement in sleep stage accuracy after applying smoothing algorithm (Liang et al., 2012).

### Discussion of Results

Selection of Features: The AASM recommends that to score sleep stages accurately, EEG leads O1-A2, C3-A2, and Fp1-A2 along with one EOG lead and one chin EMG are needed. In contrary to the recommendation, we show and suggest that only two EEG leads are sufficient for classifying sleep stages. In one embodiment, it began by extracting the EEG signal in the frontal lead (F3-A2) and the occipital lead (O1-A2). These signals are divided into 30-second epochs, the time stated by the AASM to score sleep stages in. For each epoch, we applied the CWT on the EEG signal, then sum up the coefficients within frequency bands to extract the features. In total, six features are extracted from each epoch. Since there are two leads, each epoch has twelve features extracted in total. Typically, approaches for computer algorithms used in sleep staging use either many features or very few features. While using too little features can fail to infer the actual sleep stages, using too much features can make the system prone to overfitting. Further research to explore the effect and optimize the use of less features may be needed, as they can significantly reduce the computing time and computing power, especially when considering that the purpose of the algorithm is to replace human scorers in term of time and cost.

Choice of Wavelets: The Morlet mother wavelet is a very common wavelet choice for the CWT and is available for implementation in both Python and MATLAB. However, we theorize that the shape of the Morlet mother wavelet means that only certain sleep stages are more likely to be classified correctly than others. To understand this, recall that CWT is a series of sliding and scaling the mother wavelet over a one-dimensional signal to produce coefficients. If the mother wavelet is similar to the features within the signal, then the coefficient is high. The shape of the Morlet mother wavelet is most similar to the shape of the delta and alpha waves, which means stages N3 and W are more likely to be classified correctly compared to other stages. Therefore, a direction with which to take our research would be to derive custom mother wavelets that can better classify stages N1, N2, and REM.

Unsupervised Learning: Most existing methods of sleep stages classification are supervised using trained datasets from professionals. However, the trained datasets suffer a selection bias. It is consistently shown that human professionals have low intra- and inter-patient reliability. As such, using professionals as standard for training can lead to flawed machine-learning systems. Hence, the invention develops new methods that can automatically learn the number of different sleep stages and assign which stages correspond to the actual AASM classification. This observation that unsupervised learning is possibly comes from visual examination of the features scatterplot.

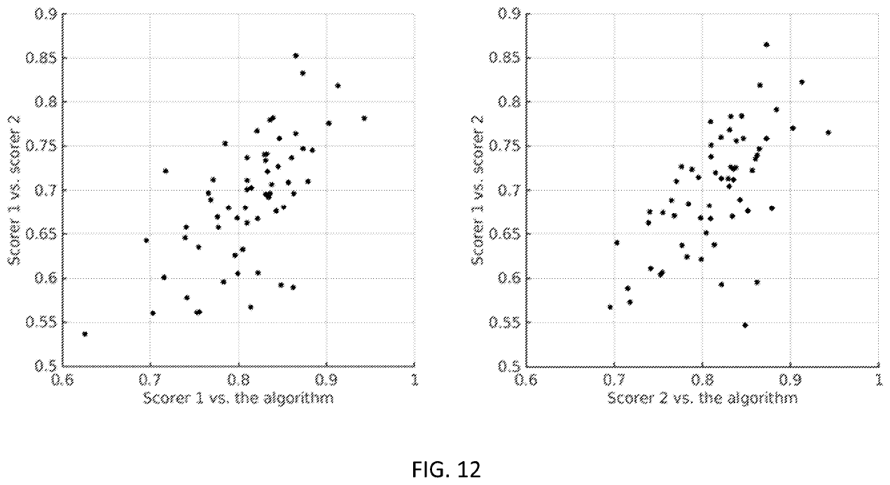

Replacement of Human Scorer: FIG. 12 contains two scatter plots showing the relationship between the scorers' agreements with each other's against the scorers' agreements with the scoring algorithm. In the left figure, we show the accuracy of the algorithm against scorer 1 on the x-axis, and the agreements between both scorers on the y-axis. In the right figure, we show the accuracy of the algorithm against scorer 2 on the x-axis, and the agreements between both scorers on the y-axis. Notice the correlation in both figures. This correlation shows that the more the scorers agree with each other, the more the scoring algorithm would agree with the scorers. If one were to draw the unity diagonal line through the scatter plot, all points would lie to the right of this line by approximately ten percent. From this observation, some insights can be gained: (1) That the algorithm's performance is similar to that of a human scorer, albeit underperformed at present by ten percent. (2) That a second professional opinion is not necessarily needed if the algorithm has high agreement with the first professional opinion. Either way, this algorithm demonstrate that it is a capable scorer of sleep stages and replace either a professional scorer or both of them.

The definite advantage of the algorithm has over the human scorer is that the algorithm is replicable over different machine configurations. The result section shows that across all 61 OSA patients and 10 healthy individuals, two professionals scoring the same sleep EEG will only agree with others 81±5% of the time. If the same algorithm were running on two different machines and score the same dataset, they would arrive at the same result, 100% of the time. Since the algorithm is under-performing compared to the professionals, we recommend the result of this algorithm be verified by at least one professional still.

In sum, aspects of the invention have demonstrated a novel method/algorithm of classifying sleep stages using the wavelet transform as the means of feature extraction and clustering as the means of classification. The algorithm performed the best when classifying stages W, N3, and REM. It performed the worst when classifying stages N1 and N2. The algorithm, while not performing as well as its human counterpart, nevertheless is very capable and replicable across different machines. This work produced promising results in replacing at least one scorer, and will be able to replace both scorers in the future.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

Al-Salman, W., Li, Y., & Wen, P. (2019). Detecting sleep spindles in EEGs using wavelet fourier analysis and statistical features. Biomedical Signal Processing and Control. https://doi.org/10.1016/j.bspc.2018.10.004
Berry, R. B., Brooks, R., Gamaldo, C. E., Harding, S. M., Marcus, C. L., & Vaughn, B. V. (2013). The AASM Manual for the Scoring of Sleep and Associated Events.
American Academy of Sleep Medicine, 53(9), 1689-1699. https://doi.org/10.1017/CBO9781107415324.004
Danker-Hopfe, H., Anderer, P., Zeitlhofer, J., Boeck, M., Dorn, H., Gruber, G., . . . Dorffner, G. (2009). Interrater reliability for sleep scoring according to the Rechtschaffen & Kales and the new AASM standard. Journal of Sleep Research. https://doi.org/10.1111/j.1365-2869.2008.00700.x
Khalighi, S., Sousa, T., Santos, J. M., & Nunes, U. (2016). ISRUC-Sleep: A comprehensive public dataset for sleep researchers. Computer Methods and Programs in Biomedicine. https://doi.org/10.1016/j.cmpb.2015.10.013
Lee, B. G., Lee, B. L., & Chung, W. Y. (2014). Mobile healthcare for automatic driving sleep-onset detection using wavelet-based EEG and respiration signals. Sensors (Switzerland). https://doi.org/10.3390/s141017915
Liang, S. F., Kuo, C. E., Hu, Y. H., Pan, Y. H., & Wang, Y. H. (2012). Automatic stage scoring of single-channel sleep EEG by using multiscale entropy and autoregressive models. IEEE Transactions on Instrumentation and Measurement. https://doi.org/10.1109/TIM.2012.2187242
Loomis, A. L., Harvey, E. N., & Hobart, G. A. (1937). Cerebral states during sleep, as studied by human brain potentials. Journal of Experimental Psychology. https://doi.org/10.1037/h0057431
Ocak, H. (2009). Automatic detection of epileptic seizures in EEG using discrete wavelet transform and approximate entropy. Expert Systems with Applications. https://doi.org/10.1016/j.eswa.2007.12.065
Pilcher, J. J., Ginter, D. R., & Sadowsky, B. (1997). Sleep quality versus sleep quantity: Relationships between sleep and measures of health, well-being and sleepiness in college students. Journal of Psychosomatic Research. https://doi.org/10.1016/S0022-3999(97)00004-4
Silber, M. H., Ancoli-Israel, S., Bonnet, M. H., Chokroverty, S., Grigg-Damberger, M. M., Hirshkowitz, M., Iber, C. (2007). The visual scoring of sleep in adults. Journal of Clinical Sleep Medicine.

What is claimed is:

1. A method for scoring sleep stages of a mammal subject, comprising:
extracting features from polysomnography (PSG) data;
grouping the extracted features into clusters that are assigned to different sleep stages; and
scoring the sleep stages based on the clusters,
wherein the sleep stages includes awake, non-rapid eye movement sleep divided into N1, N2, and N3 stages, and rapid eye movement (REM) sleep.

2. The method of claim 1, further comprising, prior to said extracting step, acquiring the PSG data by electroencephalogram (EEG) leads placed on predetermined positions on the mammal subject.

3. The method of claim 1, wherein the PSG data is pre-acquired by electroencephalogram (EEG) leads placed on predetermined positions on the mammal subject.

4. The method of claim 1, wherein said extracting step is performed with a continuous wavelet transform (CWT).

5. The method of claim 4, wherein said extracting step comprises:
separating each signal of the PSG data into epochs of a period of time without overlaps, wherein each signal is recorded with a corresponding EEG lead;
applying the CWT onto each epoch using a mother wavelet to obtain a CWT coefficient matrix;
dividing the obtained CWT coefficient matrix into six frequency bands; and
summing up the CWT coefficient matrix of each frequency band over the entire sleep epoch to obtain a feature corresponding to said frequency band for said EEG lead,
wherein in the end, each epoch taken from said EEG lead has six features extracted.

6. The method of claim 5, wherein said six frequency bands are 0-1 Hz, 1-2 Hz, 2-4 Hz, 4-8 Hz, 8-16 Hz, and 16-32 Hz.

7. The method of claim 5, wherein the mother wavelet is a Morlet wavelet.

8. The method of claim 5, wherein the period of time is in a range of about 20-40 seconds.

9. The method of claim 5, wherein said grouping step is performed with unsupervised learning.

10. The method of claim 9, wherein said grouping step comprises
detecting arousal or sudden movements;
identifying a wake stage;
identifying an N1 stage;
identifying REM and N2 stages; and
identifying an N3 stage.

11. The method of claim 10, wherein said detecting the arousal or sudden movements comprises
eliminating outliers using a density-based spatial clustering of applications with a noise (db-scan) algorithm.

12. The method of claim 11, wherein said eliminating the outliers comprises applying the db-scan) algorithm on the frequency features extracted from the PSG data acquired from a occipital (O1-A2) lead, with five frequency features at frequencies 2-4 Hz, 4-8 Hz, 8-16 Hz, and 16-32 Hz, respectively.

13. The method of claim 12, wherein a epsilon distance and nminpoints are adapted in the db-scan algorithm, such that when a group of minimum points are at most epsilon distance apart from each other's, the db-scan algorithm groups them into one cluster, and when there exist any points that are beyond the epsilon distance apart from any available clusters, the db-scan algorithm classifies them as the outliers, wherein the epsilon distance is a distance used to locate the points in the neighborhood of any point, and the nminpoints are the minimum number of points required to form a dense region.

14. The method of claim 13, wherein said five frequency features are z-score normalized using the epsilon distance of 0.7 and the nminpoints of 15, wherein any outliers registered from the step are scored as a wake stage and discarded before moving on to the next step.

15. The method of claim 11, wherein said identifying the wake stage is performed with hierarchical clustering using a Ward's method, by
starting with each point as a cluster of its own;
merging two clusters that are the most similar into a new cluster; and
repeating the staring and merging steps until there is only one cluster,
wherein the Ward's method groups the clusters that minimized the variance.

16. The method of claim 15, wherein said identifying the wake-sleep stage is performed with three frequency features at frequencies 4-8 Hz, 8-16 Hz, and 16-32 Hz, respectively, extracted from the PSG data acquired from the occipital (O1-A2) lead, so as to differentiate awake from sleep.

17. The method of claim 11, wherein said identifying the N1 stage is performed by
examining visually cluster groups of the N1 and N2 stages to determine if there are enough points in the N1 stage to classify it as a separated stage; and
with insufficient number of points in the N1 stage, marking any transitional points between the wake stage to the N2 stage as the N1 stage.

18. The method of claim 11, wherein said identifying the REM and N2 stages is performed with hierarchical clustering using Ward's criteria with three frequency features at frequencies 2-4 Hz, 4-8 Hz, and 8-16 Hz, respectively, extracted from the PSG data acquired from a frontal (F3-A2) lead, so as to differentiate the REM and N2 stages.

19. The method of claim 11, wherein said identifying the N3 stage is performed with hierarchical clustering using Ward's criteria four frequency features at frequencies 0-1 Hz, 1-2 Hz, 2-4 Hz, and 4-8 Hz, respectively, extracted from the PSG data acquired from the frontal (F3-A2) lead, so as to differentiate stage N3 from the other sleep stages.

20. The method of claim 11, wherein said grouping step further comprises smoothing the sleep stages to increase reproducibility of the scoring, which eliminates any outliers that are 2 or less epochs outside of their surrounding neighbors.

21. The method of claim 11, further comprising
comparing scores of the sleep stages to that given by a professional; and
calculating accuracy that is the number of true positive and true negative over the total number of observations, precision that is a ratio of correctly predicted positive observations to the total predicted positive observations, and sensitivity that is a ratio of correctly predicted positive observations to the all observations in actual class.

22. The method of claim 1, wherein the mammal subject is a human subject or a non-human subject.

23. A non-transitory tangible computer-readable medium storing instructions which, when executed by one or more processors, cause a system to perform a method for scoring sleep stages of a mammal subject, the method comprising:
extracting features from polysomnography (PSG) data;
grouping the extracted features into clusters that are assigned to different sleep stages; and
scoring the sleep stages based on the clusters,
wherein the sleep stages includes awake, non-rapid eye movement sleep divided into N1, N2, and N3 stages, and rapid eye movement (REM) sleep.

24. The non-transitory tangible computer-readable medium of claim 23, wherein said extracting step comprises:
separating each signal of the PSG data into epochs of a period of time without overlaps, wherein each signal is recorded with a corresponding EEG lead;
applying a continuous wavelet transform (CWT) onto each epoch using a mother wavelet to obtain a CWT coefficient matrix;
dividing the obtained CWT coefficient matrix into six frequency bands; and
summing up the CWT coefficient matrix of each frequency band over the entire sleep epoch to obtain a feature corresponding to said frequency band for said EEG lead,
wherein in the end, each epoch taken from said EEG lead has six features extracted.

25. The non-transitory tangible computer-readable medium of claim 24, wherein said six frequency bands are 0-1 Hz, 1-2 Hz, 2-4 Hz, 4-8 Hz, 8-16 Hz, and 16-32 Hz.

26. The non-transitory tangible computer-readable medium of claim 24, wherein the mother wavelet is a Morlet wavelet.

27. The non-transitory tangible computer-readable medium of claim 24, wherein the period of time is in a range of about 20-40 seconds.

28. The non-transitory tangible computer-readable medium of claim 24, wherein said grouping step is performed with unsupervised learning.

29. The non-transitory tangible computer-readable medium of claim 28, wherein said grouping step comprises
detecting arousal or sudden movements;
identifying a wake stage;
identifying an N1 stage;
identifying REM and N2 stages; and
identifying an N3 stage.

30. The non-transitory tangible computer-readable medium of claim 29, wherein said detecting the arousal or sudden movements comprises
eliminating outliers using a density-based spatial clustering of applications with a noise (db-scan) algorithm.

31. The non-transitory tangible computer-readable medium of claim 30, wherein said eliminating the outliers comprises applying the db-scan) algorithm on the frequency features extracted from the PSG data acquired from a occipital (O1-A2) lead, with five frequency features at frequencies 2-4 Hz, 4-8 Hz, 8-16 Hz, and 16-32 Hz, respectively.

32. The non-transitory tangible computer-readable medium of claim 31, wherein said five frequency features are z-score normalized using an epsilon distance of 0.7 and nminpoints of 15, wherein any outliers registered from the step are scored as a wake stage and discarded before moving on to the next step.

33. The non-transitory tangible computer-readable medium of claim 30, wherein said identifying the wake stage is performed with hierarchical clustering using a Ward's method, by
starting with each point as a cluster of its own;
merging two clusters that are the most similar into a new cluster; and
repeating the staring and merging steps until there is only one cluster,
wherein the Ward's method groups the clusters that minimized the variance.

34. The non-transitory tangible computer-readable medium of claim 33, wherein said identifying the wake-sleep stage is performed with three frequency features at frequencies 4-8 Hz, 8-16 Hz, and 16-32 Hz, respectively, extracted from the PSG data acquired from the occipital (O1-A2) lead, so as to differentiate awake from sleep.

35. The non-transitory tangible computer-readable medium of claim 30, wherein said identifying the N1 stage is performed by
examining visually cluster groups of the N1 and N2 stages to determine if there are enough points in the N1 stage to classify it as a separated stage; and
with insufficient number of points in the N1 stage, marking any transitional points between the wake stage to the N2 stage as the N1 stage.

36. The non-transitory tangible computer-readable medium of claim 30, wherein said identifying the REM and N2 stages is performed with hierarchical clustering using Ward's criteria with three frequency features at frequencies 2-4 Hz, 4-8 Hz, and 8-16 Hz, respectively, extracted from the PSG data acquired from a frontal (F3-A2) lead, so as to differentiate the REM and N2 stages.

37. The non-transitory tangible computer-readable medium of claim 30, wherein said identifying the N3 stage is performed with hierarchical clustering using Ward's criteria four frequency features at frequencies 0-1 Hz, 1-2 Hz, 2-4 Hz, and 4-8 Hz, respectively, extracted from the PSG data acquired from the frontal (F3-A2) lead, so as to differentiate stage N3 from the other sleep stages.

38. The non-transitory tangible computer-readable medium of claim 30, wherein said grouping step further comprises smoothing the sleep stages to increase reproducibility of the scoring, which eliminates any outliers that are 2 or less epochs outside of their surrounding neighbors.

39. The non-transitory tangible computer-readable medium of claim 30, further comprising
comparing scores of the sleep stages to that given by a professional; and calculating accuracy that is the number of true positive and true negative over the total number of observations, precision that is a ratio of correctly predicted positive observations to the total predicted positive observations, and sensitivity that is a ratio of correctly predicted positive observations to the all observations in actual class.

40. A system for scoring sleep stages of a mammal subject, comprising:
one or more computing devices comprising one or more processors; and
a non-transitory tangible computer-readable medium storing instructions which, when executed by the one or more processors, cause the one or more computing devices to perform a method for scoring sleep stages of a mammal subject, the method comprising:
extracting features from polysomnography (PSG) data;
grouping the extracted features into clusters that are assigned to different sleep stages; and
scoring the sleep stages based on the clusters,
wherein the sleep stages includes awake, non-rapid eye movement sleep divided into N1, N2, and N3 stages, and rapid eye movement (REM) sleep.

41. The system of claim 40, wherein said extracting step comprises:
separating each signal of the PSG data into epochs of a period of time without overlaps, wherein each signal is recorded with a corresponding EEG lead;
applying a continuous wavelet transform (CWT) onto each epoch using a mother wavelet to obtain a CWT coefficient matrix;
dividing the obtained CWT coefficient matrix into six frequency bands; and
summing up the CWT coefficient matrix of each frequency band over the entire sleep epoch to obtain a feature corresponding to said frequency band for said EEG lead,
wherein in the end, each epoch taken from said EEG lead has six features extracted.

42. The system of claim 41, wherein said six frequency bands are 0-1 Hz, 1-2 Hz, 2-4 Hz, 4-8 Hz, 8-16 Hz, and 16-32 Hz.

43. The system of claim 41, wherein the mother wavelet is a Morlet wavelet.

44. The system of claim 41, wherein the period of time is in a range of about 20-40 seconds.

45. The system of claim 41, wherein said grouping step is performed with unsupervised learning.

46. The system of claim 45, wherein said grouping step comprises
detecting arousal or sudden movements;
identifying a wake stage;
identifying an N1 stage;
identifying REM and N2 stages; and
identifying an N3 stage.

47. The system of claim 46, wherein said detecting the arousal or sudden movements comprises
eliminating outliers using a density-based spatial clustering of applications with a noise (db-scan) algorithm.

48. The system of claim 47, wherein said eliminating the outliers comprises applying the db-scan) algorithm on the frequency features extracted from the PSG data acquired from a occipital (O1-A2) lead, with five frequency features at frequencies 2-4 Hz, 4-8 Hz, 8-16 Hz, and 16-32 Hz, respectively.

49. The system of claim 48, wherein said five frequency features are z-score normalized using an epsilon distance of 0.7 and nminpoints of 15, wherein any outliers registered from the step are scored as a wake stage and discarded before moving on to the next step.

50. The system of claim 47, wherein said identifying the wake stage is performed with hierarchical clustering using a Ward's method, by
starting with each point as a cluster of its own;
merging two clusters that are the most similar into a new cluster; and
repeating the staring and merging steps until there is only one cluster,
wherein the Ward's method groups the clusters that minimized the variance.

51. The system of claim 50, wherein said identifying the wake-sleep stage is performed with three frequency features at frequencies 4-8 Hz, 8-16 Hz, and 16-32 Hz, respectively, extracted from the PSG data acquired from the occipital (O1-A2) lead, so as to differentiate awake from sleep.

52. The system of claim 47, wherein said identifying the N1 stage is performed by
examining visually cluster groups of the N1 and N2 stages to determine if there are enough points in the N1 stage to classify it as a separated stage; and
with insufficient number of points in the N1 stage, marking any transitional points between the wake stage to the N2 stage as the N1 stage.

53. The system of claim 47, wherein said identifying the REM and N2 stages is performed with hierarchical clustering using Ward's criteria with three frequency features at frequencies 2-4 Hz, 4-8 Hz, and 8-16 Hz, respectively, extracted from the PSG data acquired from a frontal (F3-A2) lead, so as to differentiate the REM and N2 stages.

54. The system of claim 47, wherein said identifying the N3 stage is performed with hierarchical clustering using Ward's criteria four frequency features at frequencies 0-1 Hz, 1-2 Hz, 2-4 Hz, and 4-8 Hz, respectively, extracted from the PSG data acquired from the frontal (F3-A2) lead, so as to differentiate stage N3 from the other sleep stages.

55. The system of claim 47, wherein said grouping step further comprises smoothing the sleep stages to increase reproducibility of the scoring, which eliminates any outliers that are 2 or less epochs outside of their surrounding neighbors.

56. The system of claim 47, further comprising
comparing scores of the sleep stages to that given by a professional; and
calculating accuracy that is the number of true positive and true negative over the total number of observations, precision that is a ratio of correctly predicted positive observations to the total predicted positive observations, and sensitivity that is a ratio of correctly predicted positive observations to the all observations in actual class.
